(19) United States
(12) Patent Application Publication
(10) Pub. No.: US 2004/0003420 A1
(43) Pub. Date: Jan. 1, 2004

(54) Modified recombinase

(76) Inventors: Ralf Kuhn, Koln (DE); Susanne Felder, Neuss (DE); Frieder Schwenk, Koln (DE); Birgit Kuter-Luks, Koln (DE); Nicole Faust, Rosrath (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 10/014,099

(22) Filed: Nov. 12, 2001

Related U.S. Application Data
(60) Provisional application No. 60/252,191, filed on Nov. 21, 2000.
(60) Provisional application No. 60/311,876, filed on Aug. 13, 2001.

(30) Foreign Application Priority Data
Nov. 10, 2000 (EP) 0012462.7
Apr. 17, 2001 (EP) 001109543

(51) Int. Cl. A01K 67/027; C07H 21/04; C12P 21/04; C12N 9/22; C12N 5/06

(52) U.S. Cl. 800/14; 435/69.7; 435/199; 435/325; 435/320.1; 536/23.2

(57) ABSTRACT

The present invention concerns a fusion protein comprising a recombinase protein, preferably the site-specific DNA recombinase C31-Int of phage φC31, and a peptide sequence which directs the nuclear uptake of the fusion protein in eucaryotic cells, and the use of this fusion protein to recombine, invert or delete DNA molecules containing recognition sequences for said recombinase in eucaryotic cells at high efficiency. In addition the invention relates to a cell, preferably a mammalian cell which contains recognition sequences for said recombinase in its genome and wherein the genome is recombined by the action of said fusion protein. Moreover, the invention relates to the use of said cell to study the function of genes and for the creation of transgenic organisms to study gene function at various developmental stages, including the adult. In conclusion, the present invention provides a process which enables the highly efficient modification of the genome of mammalian cells by site-specific recombination.

The present invention concerns a fusion protein comprising a recombinase protein, preferably the site-specific DNA recombinase C31-Int of phage φC31, and a peptide sequence which directs the nuclear uptake of the fusion protein in eucaryotic cells, and the use of this fusion protein to recombine, invert or delete DNA molecules containing recognition sequences for said recombinase in eucaryotic cells at high efficiency. In addition the invention relates to a cell, preferably a mammalian cell which contains recognition sequences for said recombinase in its genome and wherein the genome is recombined by the action of said fusion protein. Moreover, the invention relates to the use of said cell to study the function of genes and for the creation of transgenic organisms to study gene function at various developmental stages, including the adult. In conclusion, the present invention provides a process which enables the highly efficient modification of the genome of mammalian cells by site-specific recombination.

### BACKGROUND OF THE INVENTION

The controlled and permanent modification of the genome of eucaryotic cells and organisms is an important method for research applications, e.g. for studying gene function, for medical applications like gene therapy and the creation of disease models and for the design of economically important animals and crops. The basic methods for genome manipulations by the engineering of endogenous genes through gene targeting in murine embryonic stem (ES) cells are well established and used since many years (Capecchi, Trends in Genetics, 5, 70-76 (1989)). Since ES cells can pass mutations induced in vitro to transgenic offspring in vivo it is possible to analyse the consequences of gene disruption in the context of the entire organism. Thus, numerous mouse strains with functionally inactivated genes (“knock-out mice”) have been created by this technology and utilised to study the biological function of a variety of genes (Koller et al., Ann. Rev. Immunol., 10, 705-730 (1992)). More importantly, mouse mutants created by this procedure (also known as “conventional, complete or classical mutants”), contain the inactivated gene in all cells and tissues throughout life. Thus, classical mouse mutants represent the best animal model for inherited human diseases as the mutation is introduced into the germline but are not the optimal model to study gene function in adults, e.g. to validate potential drug target genes.

A refined method of targeted mutagenesis, referred to as conditional mutagenesis, employs the Cre/loxP site-specific recombination system which enables the temporally and/or spatially restricted inactivation of target genes in cells or mice (Rajewsky et al., J. Clin. Invest., 98, 600-603 (1996)). The phage P1 derived Cre recombinase recognises a 34 bp sequence referred to as loxP site which is structured as an inverted repeat of 13 bp separated by an asymmetric 8 bp sequence which defines the direction of the loxP site. If two loxP sites are located on a DNA molecule in the same orientation the intervening DNA sequence is excised by Cre recombinase from the parental molecule as a closed circle leaving one loxP site on each of the reaction products (Kilby et al., TIG, 9, 413-421 (1993)). The creation of conditional mouse mutants initially requires the generation of two mouse strains, one containing two or more Cre recombinase recognition (loxP) sites in its genome while the other harbours a Cre transgene. The former strain is generated by homologous recombination in ES cells as described above, except that the exon(s) of the target gene is (are) flanked by two loxP sites which reside in introns and do not interfere with gene expression. The Cre transgenic strain contains a transgene whose expression is either constitutively active in certain cells and tissues or is inducible by external agents, depending on the promoter region used. Crossing of the loxP-flanked mouse strain with the Cre recombinase expressing strain enables the deletion of the loxP-flanked exons in the genome of doubly transgenic offspring in a prespecified temporally and/or spatially restricted manner. Thus, the method allows the analysis of gene function in particular cell types and tissues of otherwise widely expressed genes. Moreover, it enables the analysis of gene function in the adult organism by circumventing embryonic lethality which is often the consequence of complete (germline) gene inactivation. For pharmaceutical research, aiming to validate the utility of genes and their products for drug development, gene inactivation which is inducible in adults provides an excellent genetic tool as this mimicks the biological effects of target inhibition upon drug application.

Since the first description of the concept of conditional gene targeting using the Cre/loxP system in mice in 1994 (Gu et al., Science 265, 103-106 (1994)) this method became increasingly popular among the research community and resulted in a broad collection of genetic tools for biological research in the mouse. More than 30 Cre transgenic mouse strains with various tissue specificities for gene inactivation have been created, including several “deleter” strains which allow to remove the loxP-flanked target gene segment in the male or female germine (Cohen-Tannoudji et al., Mol. Hum. Reprod. 4, 929-938 (1998); Metzger et al., Curr. Op. Biotech., 10, 470-476 (1999)). The need to characterise the expression pattern of Cre mediated recombination in newly generated strains stimulated the construction of a number of “Cre-reporter” strains which harbour a silent reporter gene the expression of which is activated upon Cre-mediated deletion (Nagy, Genesis, 26, 99-109 (2000)). Conditional mouse mutants have been reported for about 20 different genes, many of them could not be studied in adults as their complete inactivation leads to embryonic lethality (Cohen-Tannoudji et al., Mol. Hum. Reprod. 4, 929-938 (1998)).

Great efforts have also been made to control the expression of Cre recombinase in an inducible fashion in mice. After the first demonstration that inducible gene knock-out is feasible in adult mice using an interferon controlled promoter (Kühn et al., Science, 269, 1427-1429 (1995)), mainly two methods were applied to control the activity of Cre recombinase. First, it has been demonstrated that the fusion of Cre with the ligand binding domain of a mutant estrogen receptor allows to control recombinase activity by a specific steroid-like inducer. Several transgenic mouse strains expressing such a fusion protein have been generated and allow to induce gene inactivation in specific tissues (Metzger et al., Curr. Op. Biotech., 10, 470-476 (1999)). Furthermore, the tetracycline-regulated gene expression system has been successfully used to control the expression of Cre in transgenic mice and thus provides a second system for inducible gene inactivation using doxycycline as inducer (Saam et al., J. Biol. Chem. 274, 38071-38082 (1999)).

In addition to the application of Cre/loxP for gene inactivation by deletion of a gene segment this recombination system has been proved to be useful also for a number of other genomic manipulations in ES cells or mice. These include the conditional activation of transgenes in mice, chromosome engineering to obtain deletion, translocation or inversion, the simple removal of selection marker genes, gene replacement, the targeted insertion of transgenes and the (in)activation of genes by inversion (Nagy, Genesis, 26, 99-109 (2000); Cohen-Tannoudji et al., Mol. Hum. Reprod. 4, 929-938 (1998)). In conclusion, the Cre/loxP recombination system has been proven to be extremely useful for the analysis of gene function in mice by broadening the methodological spectrum for genome engineering. It can be expected that many of the protocols now established for the mouse may be applied in future also to other animals or plants.

In contrast to the huge diversity of genome manipulations which have been developed for the Cre/loxP system, very limited efforts have been made to develop further site-specific recombination systems for the use in mammalian cells. Alternative recombination systems of different specificity but with an efficiency comparable to Cre/loxP could further enhance the flexibility of genome engineering by the side to side use of two or more systems in the same cell or organism. Furthermore, unidirectional recombination systems which follow a different mechanism than the reversible Cre/loxP-mediated recombination should allow to develop new applications for genome engineering which cannot be performed with the current systems.

The reasons for the almost exclusive use of the Cre/loxP system for site-specific recombination in mammalian cells are readily explained by a number of requirements which must be fulfilled for the efficient use of a recombinase in mammalian cells:

i) the recombinase should act independent of cofactors like helper proteins,

ii) it should act independent of the supercoiling status of the target DNA and also on mammalian chromatin,

iii) it should be efficiently active and stable at a temperature of 37° C., and

iv) it should recognize a target sequence which is sufficiently long to be unique among large genomes, and it should exhibit a very high affinity to its target site for efficient action (Kilby et al., TIG, 9, 413-421 (1993)).

Among the more than 200 described members of the integrase and resolvase/invertase recombinase families only the Cre/loxP system is presently known to fulfill all of these requirements (Nunes-Düby et al., Nucleic Acids Res., 26, 391-406 (1998); Kilby et al., TIG, 9, 413-421 (1993); Ringrose et al., J. Mol. Biol., 284, 363-384 (1998)). Besides Cre/loxP a few recombinases have been shown to exhibit some activity in mammalian cells but their practical value is presently unclear as their efficieny has not been compared to the Cre/loxP system on the same genomic recombination substrate and in some cases it is known that one or more of the criteria listed above are not met. The best characterised examples are the yeast derived FLP and Kw recombinases which exhibit a temperature optimum at 30° C. but which are unstable at 37° C. (Buchholz et al., Nature Biotech., 16, 657-662 (1998); Ringrose et al., Eur. J. Biochem., 248, 903-912). For FLP it has been shown in addition that its affinity to the FRT target site is much lower as compared to the affinity of Cre to loxP sites (Ringrose et al., J. Mol. Biol., 284, 363-384 (1998)). Other recombinases which show in principle some activity in mammalian cells are a mutant integrase of phage λ, the integrases of phages φC31 and HK022, mutant γδ-resolvase and β-recombinase (Lorbach et al., J. Mol. Biol., 296, 1175-81 (2000); Groth et al., Proc. Natl. Acad. Sci. USA, 97, 5995-6000 (2000); Kolot et al., Mol. Biol. Rep. 26, 207-213 (1999); Schwikardi et al., FEBS Lett., 471, 147-150 (2000); Diaz et al., J. Biol. Chem., 274, 6634-6640 (1999)). Other phage integrase systems include coliphage P4 recombinase, Listeria phage recombinase, bacteriophage R4 Sre recombinase, CisA recombinase, XisF recombinase and transposon Tn4451 TnpX recombinase (Stark et al. Trends in Genetics 8, 432-439 (1992); Hatfull & Gridley, in Genetic Recombination. Eds. Kucherlipati & Smith, Am. Soc. Microbiol., Washington D.C., 357-396 (1988)).

However, the practical value of these recombinases and integrases for use in mammalian cells is limited as their efficiency to recombine mammalian genomic DNA has not been tested or compared with the Cre/loxP system. From the data available it can be assumed that these recombinases are much less effective than the Cre/loxP system.

In a few cases attempts have been made to improve the performance of recombinases in mammalian cells: for FLP a mutant showing improved thermostability and acticity at 37° C. has been isolated but this mutant is still considerably more heat labile as compared to Cre (Buchholz et al., Nature Biotech., 16, 657-662 (1998)). In the case of λ-integrase and γδ-resolvase the absolute requirement for coproteins and supercoiled DNA could be eliminated by the introduction of specific point mutations (Schwikardi et al. FEBS Lett 471, pp147-50 (2000)).

The import of cytoplasmic proteins into the nucleus of eucaryotic cells through nuclear pores is a regulated, energy dependent process mediated by specific receptors (Görlich et al., Science, 271, 1513-1518 (1996)). Proteins which do not posses a signal sequence recognised by the nuclear import machinery are excluded from the nucleus and remain in the cytoplasm. Numerous of such nuclear localisation signal sequences (NLS), which share a high proportion of basic amino acids in common, have been characterised (Boulikas, Crit. Rev. Eucar. Gene Expression, 3, 193-227 (1993)), the prototype of which is the 7 amino acid NLS derived from the T-antigen of the SV40 virus (Kalderon et. al, Cell, 39, 499-509 (1984)).

It was believed that the fusion of such an NLS peptide to a recombinase possibly would enhance the efficiency of the recombinase by mediating its import into the nucleus and therewith increasing the concentration of the recombinase inside the nucleus. However, for Cre recombinase it has been shown that the addition of the SV-40 T-antigen NLS does not improve its recombination efficiency in mammalian cells (Le et al., Nucleic Acid Res., 27, 4703-4709 (1999)). Nevertheless, both Cre and a Cre-NLS-fusion protein are widely used. Schwikardi (Schwikardi et al., FEBS Lett. 471, pp147-50 (2000)) reported a γδ-resolvase-SV-40 T-antigen NLS fusion protein, which also did not enhance the recombination efficiency.

The level of activity exhibited by recombinases of diverse prokaryotic origin in mammalian cells may be the result of the intrinsic properties of an enzyme depending on parameters like its temperature optimum, its target site affinity, protein structure and stability, the degree of cooperativity, the stability of the synaptic complex and the dependence on coproteins or supercoiled DNA. Within the specific environment of mammalian cells the activity of a prokaryotic recombinase could be limited by additional factors such as a short half-life of the recombinase transcript, a short half-life of its protein, its inability to act on histone-complexed and higher order structured mammalian genomic DNA, exclusion from the nucleus or the recognition of cryptic splice sites within its mRNA resulting in a nonfunctional transcript. Due to the lack of information on the parameters listed above for almost all recombinases it is presently not possible to rationally optimise their performance in mammalian cells.

### SUMMARY OF THE INVENTION

The object to be solved by the invention of the present application is the provision of a recombination system alternative to the Cre/loxP system, which has a different specificity but an efficiency comparable to Cre/loxP. Such an alternative recombination system is particularly desirable for all those applications which require more than one potent recombination system for being successfully carried out (e.g. the methods disclosed in PCT/EP01/00060 and PCT/EP00/10162). Most surprisingly, it was found that the above object can be solved by fusing a signal peptide capable directing the nuclear import (hereinafter shortly referred to as nuclear localisation signal sequences (NLS)) to specific recombinases.

In contrast to the wildtype recombinases, the resulting modified recombinases allow a highly efficient recombination of extrachromosomal and chromosomal DNA in mammalian cells, and a highly efficient excision of extrachromosomal and chromosomal DNA-stretches, which are flanked by suitable recognition sites for said modified recombinases.

The present invention thus provides:

(1) A fusion protein (hereinafter also referred to as “modified recombinase”) comprising

(a) a recombinase domain comprising a recombinase protein or fragment thereof and

(b) a signal peptide domain being linked to (a) and directing the nuclear import of said fusion protein in eucaryotic cells,

preferably the activity of the fusion protein in eucaryotic cells is significantly higher as compared to the acitivity of the wildtype recombinase corresponding to the recombinase of the recombinase domain;

(2) in a preferred embodiment of the fusion protein defined in (1) above, the recombinase domain comprises an integrase protein, preferably a phage φC31 integrase (C31-Int) protein or a mutant thereof;

(3) a DNA coding for the fusion protein as defined in (1) or (2) above;

(4) a vector containing the DNA as defined in (3) above;

(5) a microorganism containing the DNA of (3) above and/or the vector of (4) above;

(6) a process for preparing the fusion protein as defined in (1) or (2) above which comprises culturing a microorganism as defined in (5) above;

(7) the use of the fusion protein as defined in (1) or (2) above to recombine DNA molecules, which contain recombinase recognition sequences for the recombinase protein of the recombinase domain, in eucaryotic cells;

(8) a cell, preferably a mammalian cell containing the DNA sequence of (3) above in its genome;

(9) the use of the cell of (8) above for studying the function of genes and for the creation of transgenic organisms;

(10) a transgenic organism, preferably a transgenic mammal containing the DNA sequence of (3) above in its genome;

(11) the use of the transgenic organism of (10) above for studying gene function at various developmental stages; and

(12) a method for recombining DNA molecules of cells or organisms containing recognition sequences for the recombinase protein of the recombinase domain as defined in (1) or (2) above, which method comprises supplying the cells or organisms with a fusion protein as defined in (1) or (2) above, or with a DNA sequence of (3) above and/or a vector of (4) above which are capable of expressing said fusion protein in the cell or organism.

The present invention combines the use of prokaryotic recombinases such as the C31-Int with a eukaryotic signal sequence which increases its efficiency in mammalian cells such that it is equal to the widely used Cre/loxP recombination system. The improved recombination system of the present invention provides an alternative recombination system for use in mammalian cells and organisms which allows to perform the same types of genomic modifications as shown for Cre/loxP, including conditional gene inactivation by recombinase-mediated deletion, the conditional activation of transgenes in mice, chromosome engineering to obtain deletion, translocation or inversion, the simple removal of selection marker genes, gene replacement, the targeted insertion of transgenes and the (in)activation of genes by inversion.

### SHORT DESCRIPTION OF FIGURES

FIG. 1: C31-Int and Cre recombinase expression vectors and a recombinase reporter vector used for transient and stable transfections

FIG. 2: Results of transient transfections of C31 Int and Cre expression vectors and reporter vectors into CHO cells.

FIG. 3: Results of transient transfections of XisA and Ssv recombinase expression vectors with and without nuclear localisation signals and reporter vectors into CHO cells.

FIG. 4: Results of transient transfections of C31-Int and Cre recombinase vectors into a stable reporter cell line.

FIG. 5: In situ detection of β-galactosidase in 3T3(pRK64)-3 cells transfected with recombinase expression vectors

FIG. 6: Test vector for C31-Int mediated deletion, pRK64, and the expected deletion product.

FIG. 7: PCR products generated with the primers P64-1 and P64-4 and sequence comparison.

FIG. 8: ROSA26 locus of the C31 reporter mice carrying a C31 reporter construct.

FIG. 9: In situ detection of β-galactosidase in a cryosection of the testis of: (A) a double transgenic mouse carrying both the recombinase and the reporter; and (B) a transgenic mouse carrying only the reporter as a control.

### DETAILED DESCRIPTION OF THE INVENTION

The “organisms” according to the present invention are multi-cell organisms and can be vertebrates such as mammals (humans and non-human animals including rodents such as mice or rats) or non-mammals (e.g. fish), or can be invertebrates such as insects or worms, or can be plants (higher plants, algi or fungi). Most preferred living organisms are mice and fish.

“Cells” and “eucaryotic cells” according to the present invention include cells isolated from the above defined living organism and cultured in vitro. These cells can be transformed (immortalized) or untransformed (directly derived from the living organism; primary cell culture).

“Microorganism” according to the present invention relates to procaryotes (e.g. E. coli) and eucaryotic microorganisms (e.g. yeasts).

According to embodiment (1) of the present invention, the activity of the fusion protein in eucaryotic cells is significantly higher as compared to the acitivity of the wildtype recombinase corresponding to the recombinase of the recombinase domain. A “significantly higher activity” in accordance with the present invention refers to an increase in activity of at least 50%, preferably at least 75%, more preferably at least 100% relative to the corresponding wildtyp recombinase in eucaryotic cells. A “significantly higher activty” also implies that the resulting fusion protein has at least 25%, preferably at least 50% and more preferably at least 75%, of the activity of Cre/loxP in 3T3 cells with a stably integrated target sequence.

Recombinase proteins which can be used in the recombinase domain of the fusion protein of the present invention (i.e., giving a fusion having a “significantly higher activty” as defined above) include, but are not limited to, a certain type of recombinases belonging to the family of of large serine recombinases (Thorpe et al., Control of directionalty in the site-specific recombination system of the streptomyces phage φC31, Molecular Microbiology 38(2), 232-241 (2000)). This family includes bacteriophage φC31 integrase (“C31-Int”; the amino acid sequence of said integrase and a DNA sequence coding therefor are shown in SEQ ID NOs:21 and 20, respectively), coliphage P4 recombinase, Listeria phage recombinase, bacteriophage R4 Sre recombinase (“R4 Sre” deposited under GI 793758; the amino acid sequence of said recombinase and a DNA sequence coding therefor are shown in SEQ ID NOs:55 and 54, respectively), bacillus subtilis CisA recombinase (“CisA” deposited under GI 142689; the amino acid sequence of said recombinase and a DNA sequence coding therefor are shown in SEQ ID NOs:57 and 56, respectively), XisF recombinase from annabaena sp. Strain PCC 7120 (Cyanobacterium; “XisF” deposited under GI 349678; the amino acid sequence of said integrase and a DNA sequence coding therefor are shown in SEQ ID NOs:59 and 58, respectively), transposon Tn4451 TnpX recombinase (“TnpX” deposited under GI 551135; the amino acid sequence of said recombinase and a DNA sequence coding therefor are shown in SEQ ID NOs:61 and 60, respectively), “XisA” recombinase from annabaena sp. Strain PCC 7120 (Cyanobacterium; the amino acid sequence of said recombinase and a DNA sequence coding therefor are shown in SEQ ID NOs:63 and 62, respectively), “SSV” recombinase from phage of sulfolobus shibatae (the amino acid sequence of said recombinase and a DNA sequence coding therefor are shown in SEQ ID NOs:65 and 64, respectively), lactococcal bacteriophage TP901-1 recombinase (TP901-1 complete genome deposited under GI 13786531; the amino acid sequence of said recombinase and a DNA sequence coding therefor are shown in SEQ ID NOs:108 and 107, respectively), and the like, or mutants thereof. Other procaryotic recombinases known in the art are also applicable.

A “mutant” of the above recombinases in accordance with the present invention relates to a mutant of the respective original (viz. wild-type) recombinase having a recombinase activity similar (e.g. at least about 90%) to that of said wild-type recombinase. Mutants include truncated forms of the recombinase (such as N- or C-terminal truncated recombinase proteins), deletion-type mutants (where one or more amino acid residues or segments having more than one continuous amino acid residue have been deleted from the primary sequence of the wildtyp recombinase), replacement-type mutants (where one or more amino acid residues or segments of the primary sequence of the wildtyp recombinase have been replaced with alternative amino acid residues or segments), or combinations thereof.

According to embodiment (2) of the invention, the recombinase domain comprises an integrase protein, preferably a phage φC31 integrase (C31-Int) protein or a mutant thereof. Thus, the present invention provides a fusion protein comprising

(a) an integrase domain being a C31-Int protein or a mutant thereof, and

(b) a signal peptide domain being linked to (a) and directing the nuclear import of said fusion protein into eucaryotic cells.

In the fusion protein of embodiment (2), the integrase domain is preferably a C31-Int having the amino acid sequence shown in SEQ ID NO:21 or a C-terminal truncated form thereof. Suitable truncated forms of the C31-Int comprise amino acid residues 306 to 613 of SEQ ID NO:21.

The signal peptide domain (hereinafter also referred to as “NLS”) is preferably derived from yeast GAL4, SKI3, L29 or histone H2B proteins, polyoma virus large T protein, VP1 or VP2 capsid protein, SV40 VP1 or VP2 capsid protein, Adenovirus E1a or DBP protein, influenza virus NS1 protein, hepatitis virus core antigen or the mammalian lamin, c-myc, max, c-myb, p53, c-erbA, jun, Tax, steroid receptor or Mx proteins (see Boulikas, Crit. Rev. Eucar. Gene Expression, 3, 193-227 (1993)), simian virus 40 (“SV40”) T-antigen (Kalderon et. al, Cell, 39, 499-509 (1984)) or other proteins with known nuclear localisation. The NLS is preferably derived from the SV40 T-antigen.

Furthermore, the signal peptide domain preferably has a length of 5 to 74, preferably 7 to 15 amino acid residues. More preferred is that the signal peptide domain comprises a segment of 6 amino acid residues wherein at least 2 amino acid residues, preferably at least 3 amino acid residues are positively charged basic amino acids. Basic amino acids include, but are not limited to, Lysin, Arginin and Histidine. Particularly preferred signal peptides are show in the following table.

| | |
|---|---|
| Organism | Sequence/(SEQ ID NO:) |

| | | |
|---|---|---|
| yeast GAL4 | MKx11CRLKKLKCSKEKPKCAKCLKx5RX3KTKR | (24) |
| yeast SKI3 | IKYFKKFPKD | (25) |
| yeast L29 | MTGSKTRKHRGSGA | (26) |
| | (MTGSKHRKHPGSGA) | (27) |
| yeast histone H2B | (GKKRSKA) | (28) |
| poiyoma virus large T protein | (PKKAREDVSRKRPR) | (29) |
| polyoma virus VP1 capsid protein | (APKRKSGVSKC) | (30) |
| polyoma virus VP2 capsid protein | (EEDGPQKKKRRL) | (31) |
| SV40 VP1 capsid protein | (APTKRKGS) | (32) |
| SV40 VP2 capsid protein, | (PNKKKRK) | (33) |
| Adenovirus E1a protein | (KRPRP) | (34) |
| | (CGGLSSKRPRP) | (35) |
| Adenovirus DBP protein | (PPKKRMRRRIEPKKKKKRP) | (36) |
| influenza virus NS1 protein | (PFLDRLRRDQK) | (37) |
| | (PKQKRKMAR) | (38) |
| human laminA | (SVTKKRKLE) | (39) |
| human c-myc | (CGGAAKRVKLD) | (40) |
| | (PAAKRVKLD) | (41) |
| | (RQRRNELKRSP) | (42) |
| HUMAN max | (PQSRKKLR) | (43) |
| HUMAN c-myb | (PLLKKIKQ) | (44) |
| HUMAN p53 | (PQPKKKP) | (45) |
| HUMAN c-erbA | (SKRVAKRKL) | (46) |
| VIRAL jun | (ASKSRKRKL) | (47) |
| HUMAN Tax | (GGLCSARLHRHALLAT) | (48) |
| Mammalian glucocorticoid receptor | (RKTKKKIK) | (49) |
| HUMAN ANDROGEN RECEPTOR | (RKLKKLGN) | (50) |
| MAMMALIAN ESTROGEN RECEPTOR | (RKDRRGGR) | (51) |
| Mx proteins | (DTREKKKFLKRRLLRLDE) | (52) |
| SV40O T-antigen | (PKKKRKV) | (53) |

The most preferred signal peptide domain is that of SV40 T-antigen having the sequence Pro-Lys-Lys-Lys-Arg-Lys-Val.

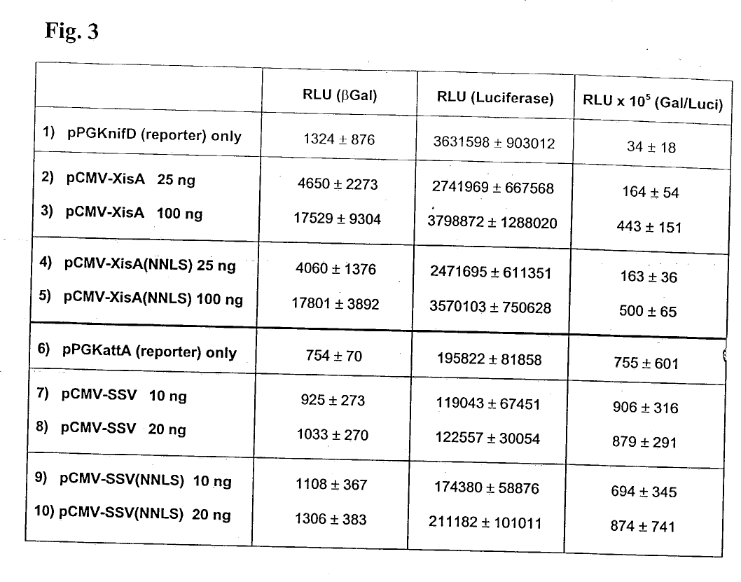

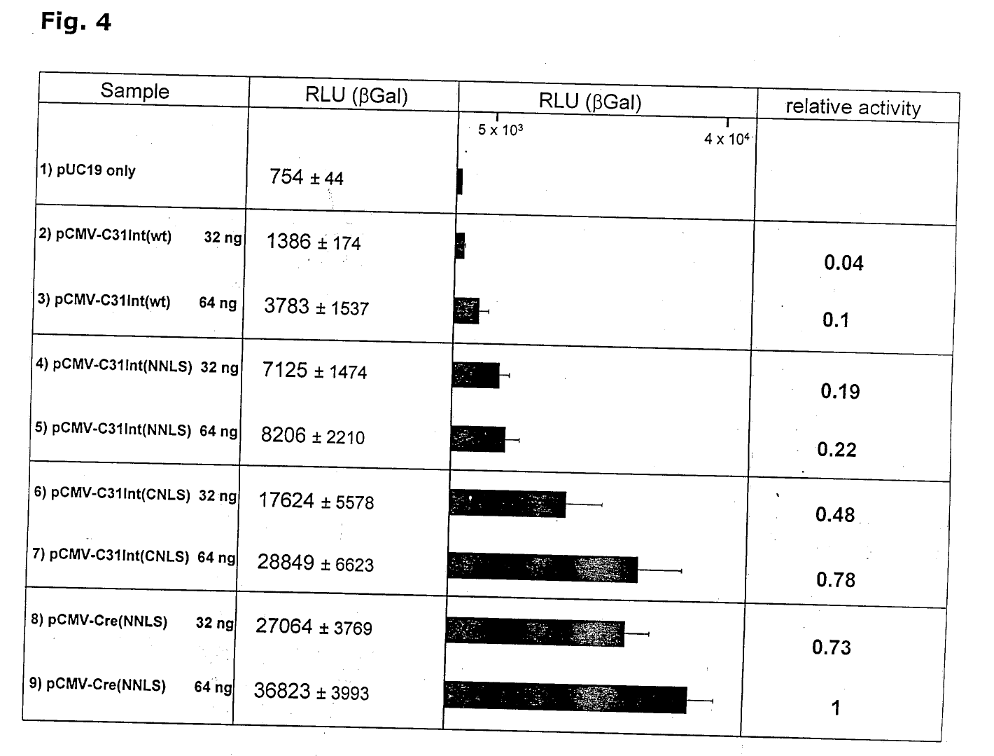

The signal peptide domain may be linked to the N-terminal or C-terminal of the integrase domain or may be integrated into the integrase domain, preferably the signal peptide domain is linked to the C-terminal of the integrase domain. With regard to phage φC31 integrase protein of embodiment (2) of the invention it was found that the fusion of an NLS-peptide to the C-terminus of the integrase provided a much higher increase of activity as compared to the fusion of the same NLS-peptide to the N-terminus of the integrase (see Example 1, FIGS. 3 and 4).

According to the present invention, the signal peptide domain may be linked to the integrase domain directly or through a linker peptide. Suitable linkers include peptides having from 1 to 30, preferably 1 to 15 amino acid residues, said amino acid residues being essentially neutral amino acids such as Gly, Ala and Val.

The most preferred fusion protein of the present invention comprises the amino acid sequence shown in SEQ ID NO:23 (a suitable DNA sequence coding for said fusion protein being shown in SEQ ID NO:22).

Further preferred fusion proteins of the present invention are “NLS-XisA” and “NLS-SSV” (having the NLS-peptide fused to the N-terminus of the recombinases) as shown in SEQ ID NO:67 and 69, respectively (suitable DNA sequences coding for said fusion proteins being shown in SEQ ID NO:66 and 68, respectively).

In embodiments (7), (8), (10) and (12) of the invention the DNA molecules, the cell or transgenic organism may also contain recognition sequences for the recombinase protein of the recombinase domain. Thus, when utilizing the fusion protein of embodiment (2), the C31-Int recognition sequences attP and attB are present in DNA molecules, the cell or transgenic organism.

The term “mammal” as used in embodiment (10) of the invention includes non-human mammals (viz. animals as defined above) and humans (if such subject matter is patentable with the respective patent authority).

Since the modified recombinase of the invention, in particular the modified C31-Int, acts in mammalian cells as efficient (or at least almost as efficient) as the widely used Cre/loxP system it can be used for a large variety of genomic modifications (including the methods disclosed in PCT/EP01/00060 and PCT/EP00/10162, the content of which is herewith incorporated by reference). Concerning embodiment (11) it is to be noted that the mammals of embodiment (10) can be used to study the function of genes, e.g. in mice, by conditional gene targeting. For this purpose suitable recognition sequences—when utilizing the fusion protein of embodiment (2), one attP and one attB site (C31-Int recognition sequences) in the same orientation—can be introduced into introns of a gene by homologous recombination of a gene targeting vector in ES cells such that the two sites flank one or more exons of the gene to be studied but do not interfere with gene expression. A selection marker gene, needed to isolate recombinant ES cell clones, can be flanked by two recognition sites of another recombinase such as loxP or FRT sites to enable deletion of the marker gene upon transient expression of the respective recombinase in ES cells. These ES cells can be used to generate germline chimaeric mice which transmit the target gene modified by att sites to their offspring and allow to establish a modified mouse strain. The crossing of this strain with a C31-Int recombinase transgenic line or the application of C31-Int protein will result in the deletion of the att-flanked gene segment from the genome of doubly transgenic offspring and the inactivation of the target gene in doubly transgenic offspring in a prespecified temporally and/or spatially restricted manner. The C31-Int transgenic strain contains a transgene whose expression is either constitutively active in certain cells and tissues or is inducible by external agents, depending on the promoter region used. If an attB and an attp site are placed into the genome in opposite orientation C31-Int mediated recombination results in the irreversible inversion of the flanked gene segment leading the functional loss of on or more exons of the target gene. Thus, the method allows the analysis of gene function in particular cell types and tissues of otherwise widely expressed genes and circumvents embryonic lethality which is often the consequence of complete (germline) gene inactivation. For the validation of genes and their products for drug development, gene inactivation which is inducible in adults provides an excellent genetic tool as this mimicks the biological effects of target inhibition upon drug application. If a pair of attB/P sites is placed in the same or opposite orientation into a chromosome at large distance using two gene targeting vectors, C31-Int recombination allows to delete or invert chromosome segments containing one or more genes, or chromosomal translocations if the two sites are located on different chromosomes. In another application of the method a pair of attB/P sites is placed in the same orientation within a transgene such that the deletion of the att-flanked DNA segment results in gene expression, e.g. of a toxin or reporter gene for cell lineage studies, or in the inactivation of the transgene.

In addition, according with embodiment (12) of the invention, the recombination system of embodiment (1), in particular the C31-Int recombination system of embodiment (2), can also be used for the site specific integration of foreign DNA into the genome of mammalian cells, e.g. for gene therapy. For this purpose, and if the C31-Int recombination system of embodiment (2) is utilized, only one attB (or attP) site is initially introduced into the genome by homologous recombination, or an endogenous genomic sequence which resembles attB or attP is used. The application of a vector containing an attP (or attB) site to such cells or mice in conjunction with the expression of C31-Int recombinase will lead to the site specific integration of the vector into the genomic att site.

Thus, the present invention provides a process which enables the highly efficient modification of the genome of mammalian cells by site-specific recombination. Said process possesses the following advantages over current technology:

(i) the modified recombinase, in particular the modified C31-Integrase, allows to recombine extrachromosomal and genomic DNA in mammalian cells at much higher efficiency as compared to the use of its wildtype form;

(ii) the modified recombinase, in particular the modified C31-Integrase, is the first described alternative recombination system with equal efficiency to Cre/loxP for the recombination of chromosomal DNA in mammalian cells.

The appended figures further explain the present invention:

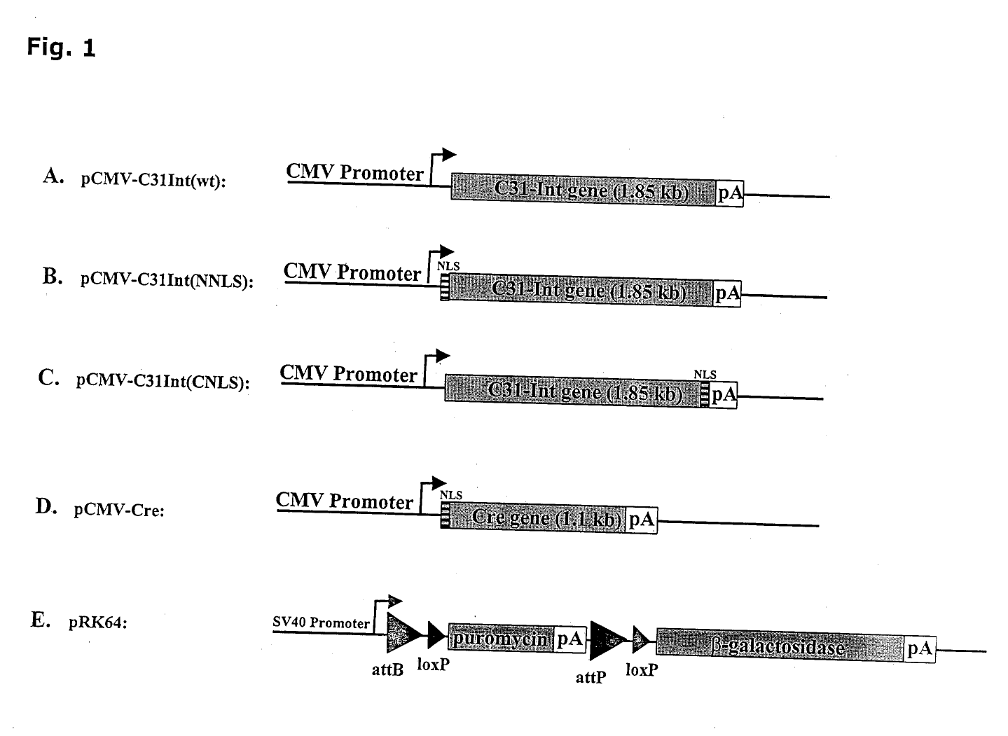

FIG. 1 shows C31-Int and Cre recombinase expression vectors and a recombinase reporter vector used for transient and stable transfections.

A-D: Mammalian expression vectors for recombinases which contain the CMV immediate early promoter followed by a hybrid intron, the coding region of the recombinase to be tested, and an artificial polyadenylation signal sequence (pA).

A: pCMV-C31Int(wt) containing the nonmodified (wildtype) 1.85 kb coding region of C31-Int as found in the genome of phage φX31.

B: pCMV-C31Int(NNLS) containing a modified C31-Int gene coding for the full length C31-Int protein with a N-terminal fusion to the SV40 virus large T antigen nuclear localisation signal (NLS).

C: pCMV-C31Int(CNLS) containing a modified C31-Int gene coding for the full length C31-Int protein with a C-terminal fusion to the SV40 virus large T antigen nuclear localisation signal (NLS).

D: pCMV-Cre contains the 1.1 kb Cre coding region with an N-terminal fusion to the SV40 T antigen NLS.

E: Recombination substrate vector pRK64 contains a SV40 promoter region followed by a 1.1 kb cassette consisting of the coding region of the puromycin resistance gene and a polyadenylation signal sequence, flanked 5′ by the 84 bp attB and 3′ by the 84 bp attP recognition site of C31-Int. pRK64 contains in addition two Cre recognition (loxP) sites in direct orientation next to the att sites.

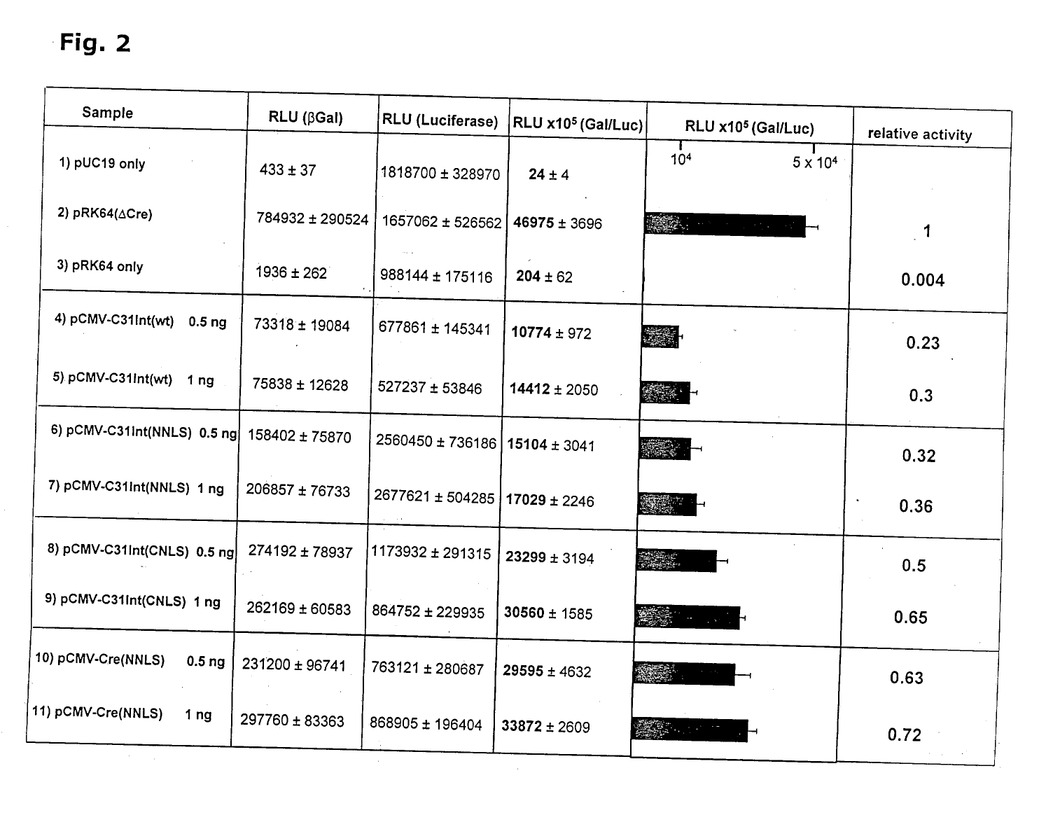

FIG. 2 shows results of transient transfections of C31-Int and Cre recombinase and reporter vectors into CHO cells.

All transfections were performed with a fixed amount of the reporter plasmid pRK64 and 0.5 ng or 1 ng of the recombinase expression plasmids pCMV-C31-Int(wt) (samples 4-5), pCMV-C31-Int(NNLS) (samples 6-7), pCMV-C31-Int(CNLS) (samples 8-9) or pCMV-Cre (samples 10-11). Negative controls: transfection with pRK64 (sample 3) or pUC19 alone (sample 1). Positive control: transfection with the Cre-recombined reporter pRK64(ΔCre) (sample 2).

The vertical rows show the mean values and standard deviation of “Relative Light Units” obtained from lysates with the assay for β-galactosidase (RLU (β-Gal)), the RLU from the assay for Luciferase, the ratio of the β-galactosidase and Luciferase values with standard deviation (RLU×10⁵ (Gal/Luc)), and the relative activity of the various recombinases as compared to the positive control defined as 1.

FIG. 3 shows results of transient transfections of XisA and Ssv recombinases and reporter vectors into CHO cells.

All transfections were performed with fixed amounts of the reporter plasmids pPGKnif (for XisA) and pPGKattA (for SSV) and 25 ng or 100 ng of the recombinase expression plasmids pCMV-XisA, pCMV-XisA(NNLS) and 10 ng or 20 ng of the expression plasmids pCMV-Ssv and pCMV-Ssv(NNLS). Negative controls: transfection with pPGKnif or pPGKattA alone.

The vertical rows show the mean values and standard deviation of “Relative Light Units” obtained from lysates with the assay for β-galactosidase (RLU (β-Gal)), the RLU from the assay for Luciferase, the ratio of the β-galactosidase and “Luciferase” values with standard deviation (RLU×10⁵ (Gal/Luc)).

FIG. 4 shows results of transient transfections of recombinase vectors into a stable reporter cell line.

All transfections were performed with a NIH 3T3 derived clone containing stably integrated copies of the pRK64 recombination substrate vector. Either 32 ng or 64 ng of the recombinase expression plasmids pCMV-C31-Int(wt) (samples 2-3), pCMV-C31-Int(NNLS) (samples 4-5), pCMV-C31-Int(CNLS) (samples 6-7) or pCMV-Cre(NNLS) (samples 8-9). Negative control: transfection with pUC19 alone (sample 1).

The vertical rows show the mean values and standard deviation of “Relative Light Units” obtained from lysates with the assay for β-galactosidase (RLU (β-Gal)) and the relative activity of the various recombinases as compared to the value obtained with pCMV-Cre(NNLS) defined as 1.

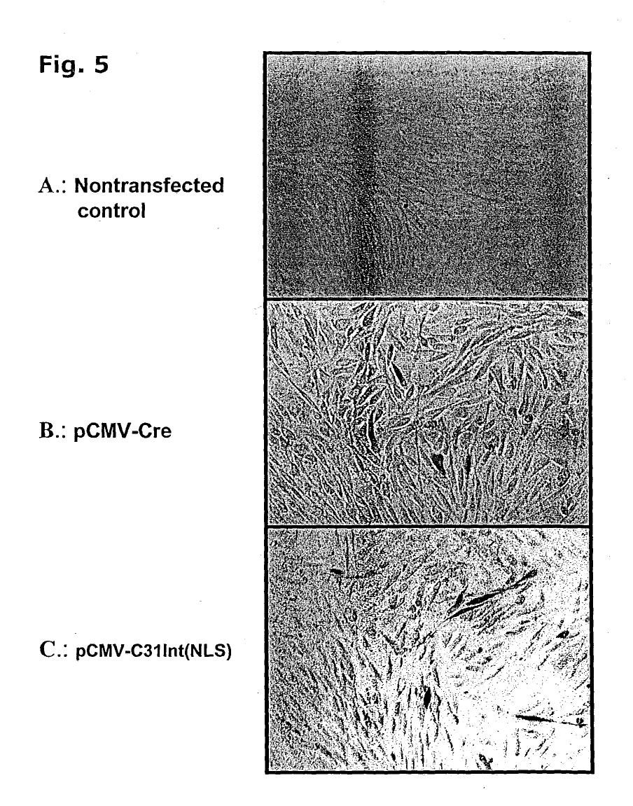

FIG. 5 shows the in situ detection of β-galactosidase in 3T3(pRK64)-3 cells transfected with recombinase expression vectors.

The Cre and C31-Int recombinase reporter cell line 3T3(pRK64)-3 was either not transfected with DNA (A), transfected with the Cre expression vector pCMV-Cre (B) or with the C31-Int expression vector pCMV-C31-Int(CNLS). Two days after tranfection the cells were fixed and incubated with the histochemical X-Gal assay which develops a blue stain in β-galactosidase positive cells indicating recombinase mediated activation of the reporter gene.

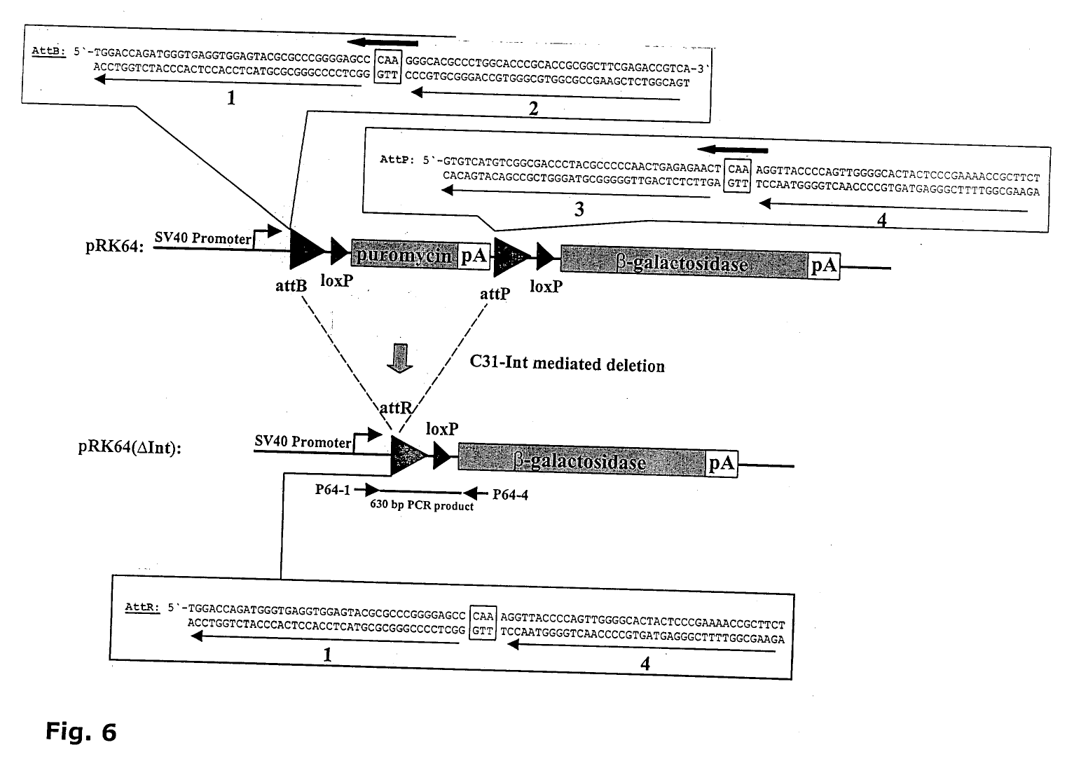

FIG. 6 shows the test vector for C31-Int mediated deletion, pRK64, and the expected product of deletion, pRK64(ΔInt).

Plasmid pRK64 contains the 1.1 kb cassette of the coding region of the puromycin resistance gene and a polyadenylation signal, which is flanked 5′ by the 84 bp attB and 3′ by the 84 bp attP recognition site (large triangles) of C31-Int. These attB and attP sites are oriented in the same way to each other (thick black arrows) which is used by the φX31 phage to integrate into the bacterial genome. In addition, the cassette is flanked by two Cre recombinase recognition (loxP) sites in the same orientation (black small triangles). For better orientation the half sites of the att sequences are labelled by a direction (thin arrow) and numbered 1-4. The 3 bp sequence within the att sites at which recombination occurs is framed by a box. The positions at which the PCR primers P64-1 and P64-4 hybridise to the pRK64 vector are indicated by arrows, pointing into the 3′ direction of both oligonucleotides.

PRK64(ΔInt) depicts the deletion product expected from the C31-Int mediated recombination between the att sites of pRK64. The recombination between a pair of attB/attP sites generates an attR site remaining on the parental DNA molecule while the puromycin cassette is excised. In this configuration the primers P64-1 and P64-4 will amplify a PCR product of 630 bp from pRK64(ΔInt).

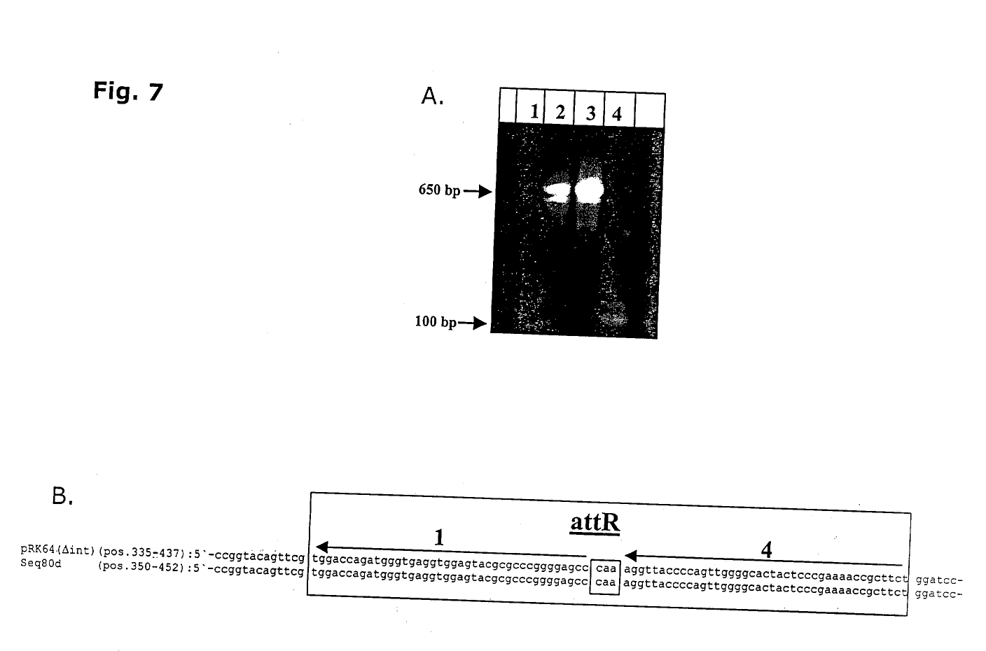

FIG. 7 shows PCR products generated with the primers P64-1 and P64-4 and a sequence comparison of the PCR product.

A: Analysis of PCR products on an agarose gel from PCR reactions using the Primers P64-1 and P64-4 on DNA extracted from MEF5-5 cells transfected 2 days before with plasmid pRK64 alone (lane 4), with pRK64+CMV-Cre (lane 3), with pRK64+pCMV-C31-Int(wt) (lane 2), and from a control reaction which did not contain cellular DNA (lane 1). The product with an apparent size around 650 bp, as compared to the size marker used, from lane 2 was excised from the agarose gel and purified. The PCR product was cloned into a sequencing plasmid vector and gave rise to the plasmid pRK80d. The insert of this plasmid was sequenced using reverse primer (seq80d) and compared to the predicted sequence of the pRK64 vector after C31-Int mediated deletion of the att flanked cassette, pRK64(ΔInt). The cloned PCR product shows a 100% identity with the predicted attR sequence after deletion. The generated attR site is shown in a box, with the same sequence designation used in FIG. 5. The nucleotide positions (pos.) of the compared sequences pRK64(ΔInt) and Seq80d are indicated.

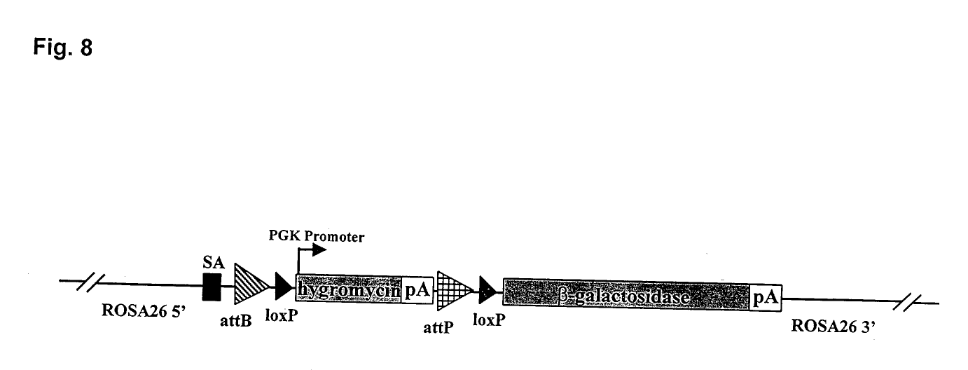

FIG. 8 shows the modified ROSA26 locus of C31 reporter mice (Seq ID NO:106). A recombination substrate has been inserted in the ROSA26 locus. The substate consists of a splice acceptor (SA) followed by a cassette consisting of the hygromycin resistance gene driven by a PGK promoter and flanked by the recombination sites attB and attp. In addition the reporter contains two Cre recognition sites (loxP) in direct orientation next to the att sites. This cassette is followed by the coding region for β-galactosidase, which is only expressed when the hygromycin resistance gene has been deleted by recombination.

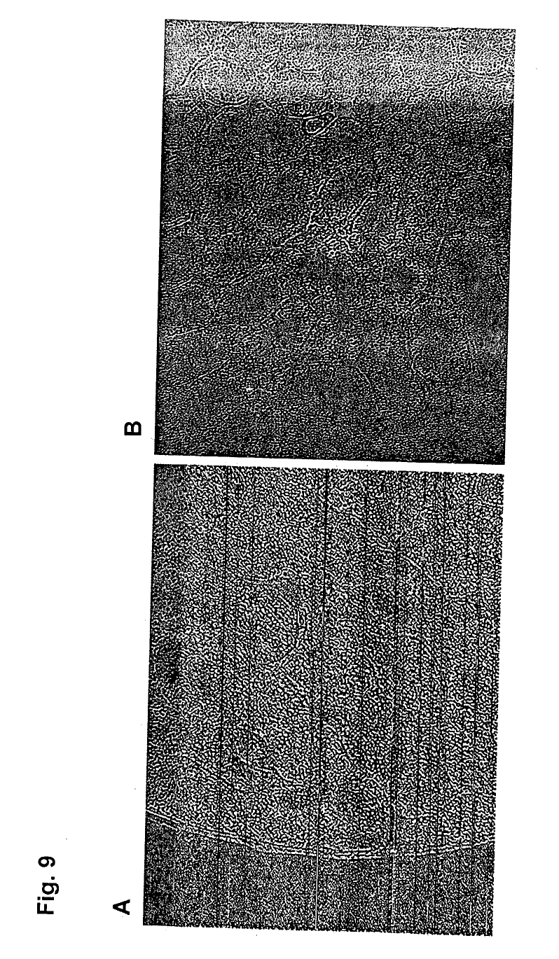

FIG. 9 shows the in situ detection of β-galactosidase activity. A cryosection of the testis of a double transgenic mouse carrying both the C31-int recombinase and the recombination substrate was stained with X-Gal (A). The blue colour indicates recombination of the substrate, which leads to the expression of β-galactosidase. As a control a cryosection of testis of a transgenic mouse carrying only the recombination substrate was stained with X-Gal (B).

The present invention is further illustrated by the following Examples which are, however, not to be construed as to limit the invention.

### EXAMPLES

### Example 1

As compared to Cre recombinase the wildtype form of C31-Int exhibits a significantly lower recombination activity in mammalian cells which falls in the range of 10-40% of Cre, depending on the assay system used (see below). As a measure which may increase C31-Int efficiency in eukaryotic cells we designed mammalian expression vectors for N- or C-terminal fusion proteins of C31-Int with a peptide was designed which is recognised by the nuclear import machinery. The recombination efficiency obtained by this modified C31-Int recombinase in mammalian cells was compared side by side to the unmodified (wildtype) form of C31-Int and to Cre recombinase. For the quantification of recombinase activities the expression vectors were transiently introduced into a mammalian cell line together with a reporter vector which contains C31-Int and Cre target sites and leads to the expression of β-galactosidase upon recombinase mediated deletion of a vector segment flanked by recombinase recognition sites.

A. Plasmid Constructions:

Construction of the recombination test vectors pPGKnif and pPGKattA: first a nifD site (Haselkorn, Annu Rev. Genet. 26, 113-130 (1992)) generated by the annealing of the two synthetic oligonucleotides nifD3 (SEQ ID NO:89) and nifD4 (SEQ ID NO:90), was ligated into the BamHI restriction site of the vector PSV-Pax1 (Buchholz et al., Nucleic Acids Res., 24, 4256-4262 (1996)), 3′ of its puromycin resistance gene and loxP site, giving rise to plasmid pPGKnifD3′ (SEQ ID NO:79). Next, another nifD site, generated by the annealing of the two synthetic oligonucleotides nifD1 (SEQ ID NO:87) and nifD2 (SEQ ID NO:88), was ligated into the BstBI restriction site of plasmid pPGKnifD3′, upstream of the puromycin resistance gene and loxP site, giving rise to plasmid pPGKnifD (SEQ ID NO:78). For pPGKattA (Muskhelishvili et al., Mol. Gen. Genet. 237, 334-342 (1993)) first a 352 bp-fragment was amplified from genomic DNA from the thermophilic bacterium Sulfolobus shibatae (DSM-5389, DSMZ. Braunschweig-Deutsche Sammlung von Mikroorganismen und Zellkulturen GmbH, Mascheroder Weg 1b, D-38124 Braunschweig, Germany) with oligonucleotides SSV5 (SEQ ID NO:96) and SSV6 (SEQ ID NO:97) including restriction sites for BamHI and BstBI. The amplified fragment was cloned into the BamHI site of the vector PSV-Pax1 giving rise to plasmid pPGKattA1 (SEQ ID NO:82), subsequently the same 352 bp-fragment was cloned into the BstBI site of pPGKattA1 giving rise to the plasmid pPGKattA2 (SEQ ID NO:83). The sequence and orientation of both nifD sites and attA sites was confirmed by DNA sequence analysis. In pPGKnifD/pPGKattA2 the newly cloned nifD/attA sites (positions 535-619 and 1722-1787/positions 6718-7081 and 12-363) are in the same orientation flanking the puromycin resistance gene and the SV40 early polyadenylation sequence. The nifD/attA sites are followed by loxP sites in the same orientation (positions 623-656 and 1794-1827/positions 7085-7118 and 369-402). The puromycin cassette is transcribed from the SV40 early enhancer/promoter region and followed by the coding region for E. coli β-galactosidase and the SV40 late region polyadenylation sequence.

Construction of XisA and SSV expression vectors: First the XisA gene of cyanobacterium PCC7120 was amplified by PCR from genomic DNA from Nostoc strain PCC7120 (CNCM-Collection Nationale de Cultures de Microorganismes, Institut Pasteur, Paris) using the primers XisA1 (SEQ ID NO:84) and XisA3 (SEQ ID NO:86), and XisA1 (SEQ ID NO:84) and XisA2 (SEQ ID NO:85) (with NLS). The ends of the PCR product were digested with NotI and the product was ligated into plasmid pBluescript II KS, opened with NotI, giving rise to plasmids pRK42a and pRK43 (with NNLS). The DNA sequence of the insert was determined and found to be identical to the published XisA sequence (Genbank GI:3953452) apart from four silent point mutations. The XisA gene was isolated as a 1.4 kb fragment from pRK42a and pRK43 by digestion with NotI and ligated into the generic mammalian expression vector pRK50 (see below), opened with NotI, giving rise to the XisA expression vectors pCMV-XisA (SEQ ID NO:76) and pCMV-XisA(NNLS) (SEQ ID NO:77). pCMV-XisA(wt) contains a Cytomegalovirus immediated early gene promoter (position 1-616), a 240 bp hybrid intron (position 716-953), the XisA gene (position 974-2392), and a synthetic polyadenylation sequence (position 2413-2591).

The SSV gene was amplified from genomic DNA from the thermophilic bacterium Sulfolobus shibatae (DSM-5389, DSMZ Braunschweig-Deutsche Sammlung von Mikroorganismen und Zellkulturen GmbH, Mascheroder Weg 1b, D-38124 Braunschweig, Germany) in two PCR steps because of an internal attP sequence. First, two overlapping PCR fragments were created with the oligonucleotides SSV1-1 (SEQ ID NO.91) (or SSV1-2 for the SSV(NNLS) gene) and SSV2 (SEQ ID NO:93) and oligonucleotides SSV3 (SEQ ID NO: 94) and SSV4 (SEQ ID NO:95). Using these overlapping fragments as template, a 1000 bp fragment containing the complete SSV coding sequence was amplified with primers SSV1-1 (or SSV1-2 for the SSV(NNLS) gene) and SSV4. The 5′ 620 bp-fragments of these PCR products were isolated by digestion with NotI-XhoI and cloned into vector pBluescript II KS giving rise to plasmids pRK47 and pRK48 (with NLS). The 3′ 380 bp fragment generated by XhoI-digestion was cloned into the XhoI restriction site of vector pBluescript II KS giving rise to the plasmid pBS-SSVs (SEQ ID NO:72). The 380 bp SSV-fragment was then isolated by digestion of pBS-SSVs with XhoI and ligated into pRK47 and pRK48 opened by XhoI giving rise to plasmids pBS-SSV3 (SEQ ID NO:70) and pBS-SSV4 (SEQ ID NO:71) (with NLS) containing the complete SSV gene. Sequencing of the plasmids confirmed one point mutation in both plasmids. Therefore 312 bp/91 bp fragments generated by digestion with EcoRV-SmaI/EcoRV-XhoI of another clone of pRK47 were exchanged in plasmids pBS-SSV3/pBS-SSV4. Sequences were confirmed by sequencing. The SSV gene was isolated from pRK47 and pRK48 by digestion with NotI and KpnI and ligated into the generic mammalian expression vecto pRK50 (see below), opened with NotI and SalI, giving rise to the SSV expression vectors pCMV-SSV(wt) (SEQ ID NO:74) and pCMV-SSV(NNLS) (SEQ ID NO:75).

Construction of the recombination test vector pRK64: first an attB site (Thorpe et al. Proc. Natl. Acad. Sci. USA, 95, 5505-5510 (1998)), generated by the annealing of the two synthetic oligonucleotides C31-4 (SEQ ID NO:1) and C31-5 (SEQ ID NO:2), was ligated into the BstBI restriction site of the vector PSV-Pax1 (Buchholz et al., Nucleic Acids Res., 24, 4256-4262 (1996)), 5′ of its puromycin resistance gene and loxP site, giving rise to plasmid pRK52. The sequence and orientation of the cloned attB site was confirmed by DNA sequence analysis. Next, an attP site site (Thorpe et al. Proc. Natl. Acad. Sci. USA, 95, 5505-5510 (1998)), generated by the annealing of the two synthetic oligonucleotides C31-6 (SEQ ID NO:3) and C31-7-2 (SEQ ID NO:4), was ligated into the BamHI restriction site of plasmid pRK52, downstream of the puromycin resistance gene and loxP site, giving rise to plasmid pRK64 (SEQ ID NO:5). The sequence and orientation of the attP site was confirmed by DNA sequence analysis. In pRK64 the newly cloned attB (position 348-431) and attP (position 1534-1617) sites are in the same orientation flanking the puromycin resistance gene and the SV40 early polyadenylation sequence. The attB and attP sites are followed by loxP sites in the same orientation (positions 435-469 and 1624-1658). The puromycin cassette is transcribed from the SV40 early enhancer/promoter region and followed by the coding region for E. coli β-galactosidase and the SV40 late region polyadenylation sequence.

Construction of C31-Int expression vectors: First the C31-Int gene of phage φC31 was amplified by PCR from phage DNA (DSM-49156, DSMZ-Deutsche Sammlung von Mikroorganismen und Zellkulturen GmbH, Mascheroder Weg 1b, D-38124 Braunschweig, Germany) using the primers C31-1 (SEQ ID NO:6) and C31-3 (SEQ ID NO:7). The ends of the PCR product were digested with NotI and the product was ligated into plasmid pBluescript II KS, opened with NotI, giving rise to plasmid pRK40. The DNA sequence of the 1.85 kb insert was determined and found to be identical to the published C31-Int gene (Kuhstoss et al., J. Mol. Biol. 222, 897-908 (1991)), except for an error in the stop codon. This error was repaired by PCR amplification of a 300 bp fragment from plasmid pRK40 using the primers C31-8 (SEQ ID NO:8) and C31-9 (SEQ ID NO:9), which provide a corrected Stop codon. The ends of this PCR fragment were digested with Eco471II and XhoI, the fragment was ligated into plasmid pRK40 and opened with Eco47III and XhoI to remove the fragment containing the defective stop codon. The resulting plasmid pRK55 contains the correct C31-Int gene as confirmed by DNA sequence analysis.

The C31-Int gene was isolated from pRK55 as 1.85 kb fragment by digestion with NotI and XhoI and ligated into the generic mammalian expression vector pRK50 (see below), opened with NotI and XhoI, giving rise to the C31-Int expression vector pCMV-C31-Int(wt). pCMV-C31-Int(wt) (SEQ ID NO:10) contains a 700 bp cytomegalovirus immediated early gene promoter (position 1-700), a 270 bp hybrid intron (position 701-970), the C31-Int gene (position 978-2819), and a 189 bp synthetic polyadenylation sequence (position 2831-3020).

For the construction of pCMV-C31-Int(NNLS) a 1.5 kb fragment was amplified by PCR from phage DNA using oligonucleotides C31-2 (SEQ ID NO:98) and C31-3 (SEQ ID NO:7). The ends of the PCR product were digested with NotI and the product was ligated into plasmid pBluescript II KS, opened with NotI, giving rise to plasmid pRK41 (SEQ ID NO: 99). A 1100 bp fragment generated by digestion of plasmid pRK41 with NcoI and NotI was then ligated into plasmid pRK55 (SEQ ID NO:80), opened with NcoI and NotI, giving rise to the plasmid pRK63 (SEQ ID NO:81). The C31-Int gene with N-terminal NLS was isolated as a 1.8 kb fragment from pRK63 by digestion with NotI and XhoI and ligated into the mammalian expression vector pRK50, opened with NotI and XhoI, giving rise to the C31-Int expression vector pCMV-C31-Int(NNLS). pCMV-C31-Int(NNLS) (SEQ ID NO:73) contains a 700 bp Cytomegalovirus immediated early gene promoter (position 1-700), a 270 bp hybrid intron (position 701-970), the C31-Int gene with N-terminal NLS (position 976-2838), and a 189 bp synthetic polyadenylation sequence (position 2851-3040).

For the construction of pCMV-C31-Int(CNLS), the 3′-end of the C31-Int gene was amplified from pCMV-C31-Int(wt) as a 300 bp PCR fragment using the primers C31-8 (SEQ ID NO:8) and C31-2-2 (SEQ ID NO:11). Primer C31-2-2 modifies the 3′-end of the wildtype C31-Int gene such that the stop codon is replaced by a sequence of 21 basepairs coding for the SV40 T-antigen nuclear localisation sequence of 7 amino acids (Prolin-Lysin-Lysin-Lysin-Arginin-Lysin-Valin) (Kalderon et. al, Cell, 39, 499-509 (1984)), followed by a new stop codon. The ends of this 300 bp PCR fragment were digested with with Eco47III and XhoI, the fragment was ligated into plasmid PCMV-C31-Int(wt) and opened with Eco47III and XhoI to replace the 3′-end of the wildtype C31-Int gene resulting in the plasmid pCMV-C31-Int(CNLS). The identity of the new gene segment was verified by DNA sequence analysis. pCMV-C31-Int(CNLS) (SEQ ID NO:12) contains a 700 bp cytomegalovirus immediated early gene promoter (position 12-711), a 270 bp hybrid intron (position 712-981), the modified C31-Int gene (position 989-2851), and a 189 bp synthetic polyadenylation sequence (position 2854-3043).

To generate the Cre expression plasmid pCMV-Cre (SEQ ID NO:13), the coding sequence of Cre recombinase (Sternberg et al., J. Mol. Biol., 187, 197-212 (1986)) with a N-terminal fusion of the 7 amino acid SV40 T-antigen NLS (see above) was recovered from plasmid pgk-Cre and cloned into the NotI and XhoI sites of plasmid pRK50. PRK50 (SEQ ID NO:14) is a generic expression vector for mammalian cells based on the cloning vector pNEB193 (New England Biolabs Inc, Beverly, Mass., USA). PRK50 was built by insertion into pNEB193 of a 700 bp cytomegalovirus immediated early gene (CMV-IE) promoter (position 1-700) from plasmid pIREShyg (GenBank#U89672; Clontech Laboratories Inc, Palo Alto, Calif., USA), a synthetic 270 bp hybrid intron (position 701-970), consisting of a adenovirus derived splice donor and an IgG derived splice acceptor sequence (Choi et al. Mol. Cell. Biol., 11, 3070-3074 (1991)), and a 189 bp synthetic polyadenylation sequence (position 1000-1188) build from the polyadenylation consensus sequence and 4 MAZ polymerase pause sites (Levitt et al., Genes&Dev., 3, 1019-1025 (1989); The EMBO J. 13, 5656-5667 (1994)). The positive control plasmid pRK64(ΔCre) (SEQ ID NO:15) was generated from pRK64 by transformation into the Cre expressing E. coli strain 294-Cre (Buchholz et al., Nucleic Acids Res., 24, 3118-3119 (1996)).

One of the transformed subclones was confirmed for the Cre mediated deletion of the loxP-flanked cassette by restriction mapping and further expanded. Plasmid pUC19 is a cloning vector without eukaryotic control elements used to equalise DNA amounts for transfections (GenBank#X02514; New England Biolabs Inc, Beverly, Mass., USA). All plasmids were propagated in DH5α E. coli cells (Life Technologies GmbH, Karlsruhe, Germany) grown in Luria-Bertani medium and purified with the plasmid DNA purification reagents “Plasmid-Maxi-Kit” (Quiagen GmbH, Hilden, Germany) or “Concert high purity plasmid purification system” (Life Technologies GmbH, Karlsruhe, Germany). Following purification, the plasmid DNA concentrations were determined by absorption at 260 nm and 280 nm in UVette cuvettes (Eppendorf-Netheler-Hinz GmbH, Hamburg, Germany) using a BioPhotometer (Eppendorf-Netheler-Hinz GmbH, Hamburg, Germany) and the plasmids were diluted to the same concentration; finally these were confirmed by separation of 10 ng of each plasmid on an ethidiumbromide-stained agarose gel.

B. Cell culture and transfections: Chinese hamster ovary (CHO) cells (Puck et al., J. Exp. Med., 108, 945 (1958)) were obtained from the Institute for Genetics (University of Cologne, Germany) as a population adapted to growth in DMEM medium. The cells were grown in DMEM/Glutamax medium (Life Technologies) supplemented with 10% fetal calf serum at 37° C., 10% CO₂ in humid atmosphere and passaged upon trypsinisation. One day before transfection 10⁶ cells were plated into a 48-well plate (Falcon). For the transient transfection of cells with plasmids each well received into 250 ml of medium a total amount of 300 ng supercoiled plasmid DNA complexed before with the FuGene6 transfection reagent (Roche Diagnostics GmbH, Mannheim, Germany) according to the manufacturers protocol. Each 300 ng DNA preparation (FIG. 2 sample 4 to 11) contained 50 ng of the luciferase expression vector pUHC13-1 (Gossen et al., Proc Natl Acad Sci USA., 89 5547-5551 (1992)), 50 ng of the substrate vector pRK64, 0.5 ng or 1 ng of one of the recombinase expression vectors pCMV-C31Int(wt), pCMV-C31Int(NNLS), pCMV-C31Int(CNLS) or pCMV-Cre and 199 ng or 199.5 ng of pUC19 plasmid, except for the controls which received 50 ng of pUHC13-1 together with 50 ng of pRK64 (sample 3) or pRK64(Δcre) (sample 2) and 200 ng pUC19, or 50 ng pUHC13-1 with 250 ng pUC19 (sample 1). Transfections of Ssv and XisA recombinases (FIG. 3) also contained 50 ng of the luciferase expression vector pUHC13-1, 50 ng of substrate vectors pPGKattA and pPGKnif and 10 ng or 20 ng of recombinase expression vector pCMV-SSV or pCMV-SSV(NNLS) or 25 ng or 100 ng of expression vectors pCMV-XisA/PCMV-XisA(NNLS). Plasmid pUC19 was added to a total amount of 300 ng plasmid DNA. As the C31-Int expression vectors are 15% larger in size than pCMV-Cre and the same amounts of DNA of the three plasmids were used for transfection, the samples with C31-Int vectors received 15% less plasmid molecules as compared to the samples with Cre expression vector. The β-galactosidase values from C31-Int transfected samples by this value were not corrected and thus is a slight underestimation of the calculated C31-Int activities. For each sample to be tested four individual wells were transfected. One day after the addition of the DNA preparations each well received additional 250 ml of growth medium. The cells of each well were lysed 48 hours after transfection with 100 ml lysate reagent supplemented with protease inhibitors (Roche Diagnostics). The lysates were centrifuged and 20 ml were used to determine the β-galactosidase activities using the β-galactosidase reporter gene assay (Roche Diagnostics) according to the manufacturers protocol in a Lumat LB 9507 luminometer (Berthold). To measure luciferase activity, 20 ml lysate was diluted into 250 ml assay buffer (50 mM glycylglycin, 5 mM MgCl₂, 5 mM ATP) and the “Relative Light Units” (RLU) were counted in a Lumat LB 9507 luminometer after addition of 100 ml of a 1 mM luciferin (Roche Diagnostics) solution. The mean value and standard deviation of the samples was calculated from the β-galactosidase and luciferase RLU values obtained from the four transfected wells of each sample.

C. Results: To set up an assay system for the measurement of C31-Int and Cre recombinase efficiency in mammalian cells the recombination substrate vector pRK64 shown in FIG. 1E was first constructed. pRK64 contains a SV40 promoter region for expression in mammalian cells followed by a 1.1 kb cassette which consists of the coding region of the puromycin resistance gene and a polyadenylation signal sequence. This cassette is flanked at the 5′-end by the 84 bp attB and at the 3′-end by the 84 bp attP recognition site of C31-Int (FIGS. 1 and 6). These attB and attP sites are located on the same DNA molecule and oriented in a way to each other which allows the deletion of the flanked DNA segment. The same orientation of attB and attP sites is used naturally by the φC31 phage and the bacterial genome, leading to the integration of the phage genome when both sites are located on different DNA molecules (Thorpe et al., Proc. Natl. Acad. Sci. USA, 95, 5505-5510 (1998)). To measure C31-Int and Cre recombinase activities with the same substrate vector, pRK64 contains in addition two Cre recognition (loxP) sites in direct orientation next to the att sites. Since the att/lox-flanked cassette in plasmid pRK64 is inserted between the SV40 promoter and the coding region of the β-galactosidase gene, its presence inhibits β-galactosidase expression as the SV40 promoter derived transcripts are terminated at the polyadenylation signal of the puromycin gene. Plasmid pRK64 is turned into a β-galactosidase expression vector upon C31-Int or Cre mediated deletion of the att/lox-flanked puromycin cassette since the remaining single att and loxP site do not substantially interfere with gene expression.

For the expression of recombinases a mammalian expression vector was designed which contains the CMV immediate early promoter followed by a hybrid intron, the coding region of the recombinase to be tested, and an artificial polyadenylation signal sequence. The backbone sequence of the four recombinase expression vectors shown in FIGS. 1A-D is identical to each other except for the recombinase coding region. Plasmid pCMV-C31Int(wt) (FIG. 1A) contains the nonmodified (wildtype) 1.85 kb coding region of C31-Int as found in the genome of phage φC31 (Kuhstoss, et al., J. Mol. Biol. 222, 897-908 (1991)). Plasmid pCMV-C31Int(NNLS) (FIG. 1B) contains a modified C31-Int gene coding for the full length C31-Int protein with a N-terminal extension of 7 amino acids derived from the SV40 virus large T antigen which serves as a nuclear localisation signal (NLS). Plasmid pCMV-C31Int(CNLS) (FIG. 1C) contains a C-terminal extension of 7 amino acids derived from the SV40 virus large T antigen which serves as a nuclear localisation signal (NLS). Plasmid pCMV-Cre (FIG. 1D) contains the 1.1 kb Cre coding region with an N-terminal fusion of the 7 amino acid NLS of the SV40 T-antigen. For Cre recombinase it has been shown that the N-terminal addition of the SV40 T-antigen NLS does not increase its recombination efficiency in mammalian cells (Le et al., Nucleic Acids Res., 27, 4703-4709 (1999)).

As a test system to compare the efficiency of the 4 recombinases the same amount of plasmid DNA of each of the recombinase expression vectors together with a fixed amount of the reporter plasmid pRK64 was transiently introduced into Chinese Hamster Ovary (CHO) cells. Thus, in this assay design the efficiency of the various recombinases on an extrachromosomal substrate introduced into the CHO cells was compared as a circular plasmid. Two days after transfection the cells from the various samples were lysed and the activity of β-galactosidase in the lysates was determined by a specific chemiluminescense assay and expressed in “Relative Light Units” (RLU (β-Gal)) (FIG. 2). In addition all samples contained a fixed amount of a luciferase expression vector to control for the experimental variation of cell transfection and lysis. For this purpose the lysates of each sample were also tested for luciferase activity with a specific chemiluminescense assay and the values expressed as “Relative Light Units” (RLU (Luciferase)) (FIG. 2). All transfection samples contained in addition varying amounts of the unrelated cloning plasmid pUC19 so that all samples were equalised to the same amount of plasmid DNA. As a positive control for β-galactosidase a derivative of the recombination reporter pRK64 was used in which the loxP flanked 1.1 kb cassette has been removed through Cre mediated recombination in E. coli giving rise to plasmid pRK64(ΔCre). As negative controls served samples which received the unrecombined reporter plasmid pRK64 but no recombinase expression vector as well as samples set up with the pUC19 plasmid; alone.

To determine the relative efficiency of the tested recombinases the RLU values of β-galactosidase were divided individually for each sample by the RLU values obtained for luciferase and multiplied with 10⁵. From the values of the four data points of each sample the mean value and standard deviation was calculated as an indicator of recombinase activity (Gal/Luc) (FIG. 2). The relative activity of the tested recombinases was then compared to the positive control defined as an activity of 1.

As shown in FIG. 2, the expression of Cre recombinase (samples 10 and 11) resulted in a 150 to 170-fold increase of β-galactosidase activity as compared to the negative control (sample 3), demonstrating the wide dynamic range of our test system. Each recombinase vector was tested using two different amounts of, DNA for transfection (0.5 and 1 ng/sample), which in the case of Cre resulted in 63% and 72% recombinase activity (samples 10 and 11 as compared to the positive control). These two values establish that the DNA amounts used are close to the test systems saturation for recombinase expression as the doubling of DNA amounts resulted only in a minor increase of recombinase activity.

In comparison to Cre, the expression of wildtype C31-Int resulted in a considerably lower recombinase activity of 23% and 30% (FIG. 2, samples 4 and 5) as compared to the positive control. These values represent 37% and 42% recombinase activity for wildtype C31-Int as compared to Cre recombinase (compare samples 4 and 5 with 10 and 11). Upon the expression of C31-Int fused with the N-terminal NLS (C31-Int(NNLS)) values of 32% and 36% recombinase activity (samples 6 and 7) were obtained as compared to the positive control. The C31-Int(NNLS) values represent 51% and 50% recombinase activity as compared to Cre (compare samples 6 and 7 to 10 and 11). Thus, the activity of C31-Int in mammalian cells is just moderately enhanced by the addition of a NLS signal.

Surprisingly, upon the expression of C31-Int fused with the C-terminal NLS (C31-Int(CNLS)) values of 50% and 65% recombinase activity (samples 8 and 9) were obtained as compared to the positive control. The C31-Int(CNLS) values represent 79% and 90% recombinase activity as compared to Cre recombinase (compare samples 84 and 9 to 10 and 11). Unexpectedly, C31-Int(CNLS) exhibits a dramatic, more than twofold increase of recombinase activity in comparison to C31-Int(wt) (compare samples 8 and 9 to 4 and 5).

In order to test whether the addition of a NLS sequence may be a general, simple method to enhance recombinase activity in mammalian cells we extended our studies by two additional recombinases: XisA recombinase (XisA) derived from the cyanobacterium Anabaena, and SSV-Integrase (SSV-Int) derived from the SSV1 virus of the thermophilic bacterium Sulfolobus shibatae. To this end we constructed mammalian expression vectors for the wildtype forms of XisA and SSV recombinases and compared their activity to versions which were modified by the N-terminal addition of the 7 amino acid NLS of the SV40 T-antigen. These recombinases were compared by the use of the reporter vector shown in FIG. 1E, except that the att elements of C31-Int were replaced by the nif recognition sequences for XisA or the att sequences for SSV-Int. As described above for C31-Int, recombinase activities were tested by transient transfection into CHO cells using the reporter vector derived β-galactosidase activity as readout and cotransfected luciferase as internal control.

As shown in FIG. 3 for both, XisA and SSV recombinases the addition of a NLS sequence did not improve their activity in a mammalian cell line as compared to the wildtype forms. At both DNA concentrations tested wildtype XisA exhibits a significant recombination activity as compared to the reporter vector alone (compare samples 2 and 3 to sample 1), but this activity is not altered by the addition of an NLS (compare samples 2 and 3 to samples 4 and 5). SSV-Int exhibits only a low recombination activity (compare samples 7 and 8 with sample 6) which is also not enhanced by the addition of a NLS (compare samples 9 and 10 with samples 7 and 8). From these results we conclude that the addition of a NLS to an inefficient recombinase is not a general, simple method to improve its performance in mammalian cells.

Taken together, in the transient transfection test system shown in FIG. 2 a more than twofold activity increase of the φC31 Integrase could be achieved by the C-terminal, but not the N-terminal addition of the SV40 T antigen NLS signal. As this signal sequence has been characterised to act as a nuclear localisation signal (Kalderon et. al, Cell, 39, 499-509 (1984)) we conclude that the efficiency increase of C31-Int(CNLS) is the result of the improved nuclear accumulation of this recombinase. The relative inefficiency of C31-Int (NNLS) may be explained by the inaccessibility of the NLS peptide to the nuclear import machinery at the N-terminal position of the C31-Int protein.

In particular, it could be shown that C31-Int(CNLS) recombines extrachromosomal DNA in mammalian cells almost as efficient as the widely used Cre recombinase and thus provides an additional or alternative recombination system of highest activity. The efficiency increase of C31-Int(CNLS) as compared to its wildtype form is regarded as an invention of substantial use for biotechnology.

### Example 2

As demonstrated in example 1 C31-Int recombinase with the C-terminal fusion of the SV40 T-antigen NLS (C31-Int(CNLS)) shows in mammalian cells a recombination activity comparable to Cre recombinase on an extrachromosomal plasmid vector. It was further tried to test whether C31-Int(CNLS) exhibits a similar activity on a recombination substrate which is chromosomally integrated into the genome of mammalian cells. This question is critical for the use of a recombination system for genome engineering as it is possible that a recombinase may act efficiently on extrachromosomal substrates but is impaired if the recognition sites are part of the mammalian chromatin. To characterise the recombination activity of C31-Int(CNLS) and C31-Int(NNLS) on a chromosomal substrate the pRK64 reporter plasmid (FIG. 1E) was stably integrated, containing a pair of loxP and att sites, into the genome of a mammalian cell line. One of the stable transfected clones was chosen for further analysis and was transiently transfected with recombinase expression vectors coding for C31-Int(CNLS), C31-Int(NNLS), C31-Int(wt) or Cre recombinase. The activity of β-galactosidase derived from the Cre expression vector recombined in these cells was taken as a measure of recombination efficiency.

A. Plasmid constructions: all plasmids used and their purification are described in example 1.

B. Cell culture and transfections: To generate a stably transfected C31-Int reporter cell line 2.5×10⁶ NIH-3T3 cells (Andersson et al., Cell, 16, 63-75 (1979); DSMZ#ACC59; DSMZ-Deutsche Sammlung von Mikroorganismen und Zellkulturen GmbH, Mascheroder Weg 1b, D-38124 Braunschweig, Germany) were electroporated with 5 μg pRK64 plasmid DNA linearised with ScaI and plated into 10 cm petri dishes. The cells were grown in DMEM/Glutamax medium (Life Technologies) supplemented with 10% fetal calf serum at 37° C., 10% CO₂ in humid atmosphere, and passaged upon trypsinisation. Two days after tranfection the medium was supplemented with 1 mg/ml of puromycin (Calbiochem) for the selection of stable integrants. Upon the growth of resistant colonies these were isolated under a stereomicroscope and individually expanded in the absence of puromycin. To demonstrate stable integration of the transfected vector, genomic DNA of puromycin resistant clones was prepared according to standard methods and 5-10 μg were digested with EcoRV. Digested DNA was separated in a 0.8% agarose gel and transferred to nylon membranes (GeneScreen Plus, NEN DuPont) under alkaline conditions for 16 hours. The filter was dried and hybridised for 16 hours at 65° C. with a probe representing the 5′ part of the E. coli β-galactosidase gene (1.25 kb NotI-EcoRV fragment of plasmid CMV-β-pA (R. Kühn, unpublished). The probe was radiolabelled with P32-marked α-dCTP (Amersham) using the Megaprime Kit (Amersham). Hybridisation was performed in a buffer consisting of 10% dextranesulfate, 1% SDS, 50 mM Tris and 100 mM NaCl, pH 7.5). After hybridisation the filter was washed with 2×SSC/1% SDS and exposed to BioMax MS1 X-ray films (Kodak) at −80° C.

Transfections of the selected clone 3T3(pRK64)-3 with plasmid DNAs and the measurement of β-galactosidase activities in lysates were essentially performed as described in example 1 for CHO cells, except that 32 ng or 64 ng of the recombinase expression plasmids and 218 or 186 ng of pUC19 plasmid were used and the pRK64 plasmid was omitted from all samples.

C. Histochemical Detection of β-Galactosidase Activity in Transfected 3T3(pRK64)-3 Cells

To directly demonstrate β-galactosidase expression in recombinase transfected cells, 10⁴ 3T3(pRK64)-3 cells were plated one day before transfection into each well of a 48-well tissue culture plate (Falcon). For the transient transfection of cells with plasmids each well received into 250 μl of medium a total amount of 150 ng supercoiled plasmid DNA complexed before with the FuGene6 transfection reagent (Roche Diagnostics GmbH, Mannheim, Germany) according to the manufacturers protocol. Each 150 ng DNA preparation contained 50 ng of the recombinase expression vector pCMV-Cre or pCMV-C31Int(CNLS) and 100 ng of the pUC19 plasmid. After 2 days the culture medium was removed from the wells, the wells were washed once with phosphate buffered saline (PBS), and the cells were fixed for 5 minutes at room temperature in a solution of 2% formaldehyde and 1% glutaraldehyde in PBS. Next, the cells were washed twice with PBS and finally incubated in X-Gal staining solution for 24 hours at 37° C. (staining solution: 5 mM K₃(Fe(CN)₆), 5 mM K₄(Fe(CN)₆), 2 mM MgCl₂, 1 mg/ml X-Gal (BioMol) in PBS) until photographs were taken.

D. Results

To generate a mammalian cell clone with a stable genomic integration of the C31-Int and Cre recombinase reporter plasmid pRK64, the murine fibroblast cell line NIH-3T3 was electroporated with linearised pRK64 DNA (FIG. 1D; see also example 1) and subjected to selection in puromycin containing growth medium. Plasmid pRK64 contains in between the pair of loxP and att sites the coding region of the puromycin resistance gene expressed from the SV40-IE promoter. Thirty-six puromycin resistant clones were isolated and the genomic DNA of 19 clones was analysed for the presence and copy number of the pRK64 DNA. Three clones, which apparently contain 2-4 copies of pRK64, were further characterised on the single cell level for the expression of β-galactosidase upon transient transfection with the Cre expression vector pCMV-Cre (FIG. 1C). The cell clone with the largest proportion of β-galactosidase positive cells, 3T3(pRK64)-3, was selected as most useful for the planned studies on C31-Int and Cre recombinase efficiency.

To compare the efficiency of wildtype C31-Int (C31-Int(wt)), C31-Int(CNLS), C31-Int(NNLS), and Cre recombinases 32 ng or 64 ng of the recombinase expression vectors pCMV-C31Int(wt), pCMV-C31Int(CNLS), pCMV-C31Int(NNLS), or pCMV-Cre (FIGS. 1A-D) together with the unrelated cloning plasmid pUC19 were transiently introduced into 3T3(pRK64)-3 cells, such that all samples contained the same amount of plasmid DNA. As a negative control a sample prepared with the pUC19 plasmid alone was used. Two days after transfection the cells from the various samples were lysed and the activity of β-galactosidase in the lysates was determined by a specific chemiluminescense assay and expressed in “Relative Light Units” (RLU)(β-Gal) (FIG. 4). From the values of the four data points of each sample the mean value and standard deviation was calculated as an indicator of recombinase activity (FIG. 4). The relative activity of the tested recombinases was then compared to the highest value obtained with the Cre expression vector, defined as an activity of 1.

As shown in FIG. 4 the expression of Cre recombinase (samples 8 and 9) resulted in a 36 to 49-fold increase of β-galactosidase activity as compared to the negative control (sample 1), demonstrating the dynamic range of the test system used. Each recombinase vector was tested using two different amounts of DNA for transfection (32 ng and 64 ng/sample), which in the case of Cre resulted in 73% and 100% recombinase activity (samples 8 and 9). These two values establish that the DNA amounts used are not far from the linear scale of the test systems ability for recombinase expression as the twofold increase of the amount of DNA also resulted in a significant increase of recombinase activity.

The expression of wildtype C31-Int (FIG. 4, samples 2 and 3) resulted in a low recombinase activity of 4% and 10% as compared to the values obtained by Cre transfection (compare samples 2 and 3 with 8 and 9). This activity was only moderately enhanced by the expression of C31-Int(NNLS) to values of 19% and 22% of Cre activity (compare samples 4 and 5 with samples 8 and 9). Upon the expression of C31-Int(CNLS) values of 48% and 78% recombinase activity were obtained as compared to Cre recombinase (compare samples 6 and 7 to 8 and 9). Hence, C31-Int(CNLS) exhibits an 12-fold higher activity than C31-Int(wt) at 32 ng plasmid DNA (FIG. 4, compare samples 6 and 2) and an 8-fold higher activity than C31-Int(wt) at 64 ng plasmid DNA (FIG. 4, compare samples 7 and 3).

In addition, it was aimed to directly demonstrate in situ the expression of β-galactosidase in 3T3(pRK64)-3 cells after transfection with Cre or C31-Int(CNLS) recombinase plasmid. Two days after transfection the cells were fixed in situ and incubated with the histochemical X-Gal assay which detects β-galactosidase positive cells by a blue precipitate. As shown in FIG. 5 stained cells were found at a comparable frequency in the samples transfected with the Cre or C31-Int(CNLS) expression vectors but not in the nontransfected control. This result confirms that the β-galactosidase activities measured by chemiluminescense upon recombinase transfection (FIG. 4) results from a population of individual, recombined reporter cells.

In conclusion, upon the transient transfection of recombinase expression vectors into a cell line with a genomic integration of the recombination substrate vector, a 8-12-fold activity increase of the φC31 Integrase by the C-terminal fusion with the SV40 T-antigen NLS signal was found. As this signal sequence has been characterised to act as a nuclear localisation signal (Kalderon et. al, Cell, 39, 499-509 (1984)), it was concluded that the dramatic efficiency increase of C31-Int(CNLS) is the result of the improved nuclear accumulation of this recombinase. The approximately tenfold activity increase of C31-Int(CNLS) upon expression in a cell line with a genomic integration of the substrate vector is considerably higher than the activity increase found upon the transient expression of both vectors (see example 1). Thus, a substrate vector integrated into the chromatin of a mammalian cell may pose more stringent requirements on recombinase activity to be recombined as compared to an extrachromosomal substrate.

The dramatic activity increase of C31-Int(CNLS), as compared to its wildtype form, on a stable integrated substrate in mammalian cells is an invention of significant practical use as this recombinase is as efficient as the widely used Cre/loxP system; thus, C31-Int(CNLS) provides an additional or alternative recombination system of highest activity.

### Example 3

To demonstrate that the increase in β-galactosidase activity obtained by the cotransfection of a C31-Int expression vector and the reporter vector pRK64 into mammalian cells is in fact the result of recombinase mediated deletion, one of the recombination products was detected by a specific polymerase chain reaction (PCR). The amplified PCR product was cloned and its sequence determined. The obtained sequence confirms that C31-Int mediated deletion of the test vector occurs in a mammalian cell line and that the recombination occurs at the known breakpoint within the attB and attP sites.

A. Plasmid constructions: The construction of plasmids pRK64, pCMV-Cre and pCMV-C31-Int(wt) is described in Example 1. To simulate the recombination of pRK64 by C31-Int, the sequence between the CAA motives of the att sites (boxed in FIG. 5) was deleted from the computerfile of pRK64, giving rise to the sequence of pRK64(ΔInt) (SEQ ID NO:16).

B. Transfection of Cells and PCR amplification: MEF5-5 mouse fibroblasts (Schwenk et al., 1998) (20000 cells per well of a 12 well plate (Falcon)) were transfected with 0.5 μg pRK64 alone or together with 250 ng pCMV-Int(wt) or pCMV-Cre using the FuGene6 transfection reagent following the manufacturers protocol (Roche Diagnostics). After 2 days DNA was extracted from these cells according to standard methods and used for PCR amplification with Primers P64-1 (SEQ ID NO:17; complementary to position 111-135 of pRK64(ΔInt)) and P64-4 (SEQ ID NO:18; complementary to position 740-714 of pRK64(ΔInt)) using the Expand High Fidelity PCR kit (Roche Diagnostics). PCR products were separated on a 0.8% agarose gel, extracted with the QuiaEx kit (Quiagen) and cloned into the pCR2.1 vector using the TA cloning kit (Invitrogen) resulting in plasmid pRK80d. The sequence of its insert, seq80d (SEQ ID NO:19), was determined using the reverse sequencing primer and standard sequencing methods (MWG Biotech).

For the measurement of β-galactosidase activity the cells were lysed 2 days after transfection and the β-galactosidase activities were determined with the β-galactosidase reporter gene assay (Roche Diagnostics) as described in example 1.

C. Results: As a test vector for C31-Int mediated DNA recombination plasmid pRK64 was used, which contains the 1.1 kb coding region of the puromycin resistance gene flanked 5′ by the 84 bp attB and 3′ by the 84 bp attP recognition site of C31-Int (FIG. 5). These attB and attP sites are located on the same DNA molecule and oriented in a way to each other which allows the deletion of the att-flanked DNA segment. The same orientation of attB and attP sites is used naturally by the φC31 phage and the bacterial genome for the integration of the phage genome when both sites are located on different DNA molecules (Thorpe et al., Proc. Natl. Acad. Sci. USA, 95, 5505-5510 (1998)). As a positive control, vector pRK64 contains in addition two Cre recombinase recognition (loxP) sites in direct orientation next to the att sites. Since the att-flanked DNA segment in plasmid pRK64 is inserted between a promoter active in mammalian cells and the β-galactosidase gene, its deletion can be measured by the increase of β-galactosidase activity. The expected product of C31-Int mediated deletion of plasmid pRK64 is shown in FIG. 6, designated as pRK64(ΔInt). If the recombination between attB and attP occurs as described in bacteria (Thorpe et al., Proc. Natl. Acad. Sci. USA, 95, 5505-5510 (1998)), a single attR site is generated and left on the parental plasmid (FIG. 6) while the flanked DNA is excised and contains an attL site. Beside the measurement of β-galactosidase activity, C31-Int mediated recombination of pRK64 can be directly detected on the DNA level by a specific polymerase chain reaction (PCR) using the primers P64-1 and P64-4 (FIG. 6). These primers, located 5′ of the attB site (P64-1) and 3′ of the attP site, are designed to amplify a PCR product of 630 bp lenght upon the C31-Int mediated recombination of pRK64. For the expression of C31-Int in mammalian cells plasmid pCMV-C31(wt) was used, which contains the CMV-IE-Promoter upstream of the C31-Int coding region followed by a synthetic polyadenylation signal (see Example 1 and FIG. 1).

The recombination substrate vector pRK64 was transiently transfected into the murine fibroblast cell line MEF5-5 either alone, or together with the C31-Int expression vector pCMV-C31(wt), or together with an expression vector for Cre recombinase, pCMV-Cre. Two days after transfection half the cells of each sample was lysed and used to measure β-galactosidase activity by chemiluminescense, and the other half was used for the preparation of DNA from the transfected cells for PCR analysis. The β-galactosidase levels of the 3 samples were found as following (expressed as “Relative Light Units” (RLU) with standard deviation (SD) of the β-galactosidase assay):

| Sample | RLU (SD) |
|---|---|
| 1) pRK64 | 692 ± 5 |
| 2) pRK64 + pCMV-Cre | 8527 ± 269 |
| 3) pRK64 + pCMV-C31 (wt) | 1288 ± 93 |

As the coexpression of the test vector pRK64 together with the C31-Int expression vector in sample 3 leads to a significant increase of β-galactosidase activity as compared to pRK64 alone, this result suggests that pRK64 is recombined by C31-Int as anticipated in FIG. 6.

Next, cellular DNA was prepared from the three samples and tested for the occurrence of the expected Cre or C31-Int generated deletion product by PCR using primers P64-1 and P64-4 for amplification. As shown in FIG. 7 an amplification product of the expected size was found only in the samples cotransfected with the Cre or C31-Int recombinase expression vectors (FIG. 7A, lane 3 and lane 4). The PCR products amplified from pRK64recombined by C31-Int or Cre are of the same size but should be recombined via the attB/P or loxP sites, respectively.

To prove that the PCR product found after cotransfection of plasmids pRK64 and pCMV-C31(wt) represents in fact the deletion product of C31-Int mediated recombination, this DNA fragment was cloned into a plasmid vector and its DNA sequence determined. One clone, pRK80d, was analysed, and its sequence showed exactly the sequence of an attR site as expected from C31-Int mediated deletion of pRK64 (FIG. 7B, compare to FIG. 6).

In conclusion, this experiment demonstrates that C31-Int mediated deletion of a vector containing a pair of attB/attP sites occurs in a mammalian cell line, and that the recombination occurs within the same 3 bp breakpoint region of attB and attP as found in bacteria (Thorpe et al., Proc. Natl. Acad. Sci. USA, 95, 5505-5510 (1998)). Thus, it was concluded that an increase of β-galactosidase activity observed by cotransfection of the pRK64 reporter vector and a C31-Int expression vector in mammalian cells truly reflects C31-Int recombinase activity.

### Example 4

As has been demonstrated in examples 1-3, the C31-Int recombinase with the C-terminal fusion of the SV40 T-antigen NLS (C31-Int(CNLS)) shows a recombination activity comparable to Cre recombinase on an extrachromosomal as well as a chromosomally integrated target in mammalian cells in vitro. To test whether C31-Int(CNLS) exhibits activity in mice, transgenic mice carrying a C31-Int(CNLS) expression vector were generated. These transgenic mice were crossed with reporter mice carrying the recombinase substrate. Recombination-mediated expression of β-galactosidase, which can be measured by staining with the substrate X-Gal, was analyzed in testes of double transgenic progeny carrying both the recombinase and the reporter.

A. Plasmid constructions: For the construction of the C31-Int(CNLS) transgene expression vector, the C31Int gene with C-terminal NLS was isolated as a 2 kb-fragment generated by restriction of pCMV-C31Int(CNLS) (SEQ ID NO: 12) with BglII. The fragment was ligated into the BglII restriction site of the vector pCAGGS-Cre-pA (SEQ ID NO:104) giving rise to the plasmid pCAGGS-C31CNLS-pA (SEQ ID NO:105). In pCAGGS-C31CNLS-pA the C31-Int(CNLS) (position 1891-3753) is transcribed from the CAGGS promoter (position 1-1616) and followed by the SV40 late region polyadenylation sequence (position 3763-3941).

B. Production of transgenic mice: For the embryo injections a 3.95 kb-fragment was generated by restriction of the plasmid pCAGGS-C31CNLS-pA with PstI and AscI. This fragment was purified as follows: DNA bands were separated on an agarose-gel without ethidiumbromide. One part of the gel was stained with ethidiumbromide to locate the band to excise. The DNA was electroeluted from the excised band with S&S Biotrap Elution Chamber in 1×TAE (40 mM Tris-acetate, 1 mM EDTA) overnight. The DNA was precipitated from the eluate with 1/10 volume 3M sodium acetate and 2.5 volumes ethanol at −20° C. for several hours. The DNA was pelleted by centrifugation at 13000 rpm for 30 min and washed twice with 70% ethanol. The dried DNA pellet was resuspended in TE (10 mM Tris, 1 mM EDTA, pH 8). Subsequently the precipitation procedure was repeated once and the DNA resuspended in injection buffer (10 mM Tris pH 7.2, 0.1 mM EDTA). The sample was dialysed with Slide-A-Lyse Mini Dialysis Unit (Pierce) in injection buffer with several changes of buffer at 4° C. overnight. Different amounts of the sample were checked on a gel to determine concentration. To generate transgenic mice, 5-10 fg of the purified fragment were injected into one pronucleus of (B6CBA)F2 mouse one-cell embryos. The injected embryos were subsequently transferred into the oviduct of 0.5 day pseudopregnant NMRI females.

C. Analysis of transgenic mice: Mice were analyzed for the presence of the pCAGGS-C31CNLS-pA transgene by PCR using tail DNA and the primers C31-screen 1 (SEQ ID NO:100) and C31-screen 2 (SEQ ID NO:101) amplifying a fragment of 500 bp. The PCR reaction contained 5 μl PCR buffer (Invitrogen), 2 μl 50 mM MgCl₂, 1.5 μl 10 mM dNTP-mix, 2 μl (10 pmol) of each primer, 0.5 μl Taq-polymerase (5 U/μl) and water to a volume of 50 μl. The program used for the PCR reactions was: 94° C. for 30 s, 55° C. for 30 s and 72° C. for 1 min in 30 cycles.

D. Analysis of C31-Int(CNLS) activity: Founder mice transgenic for the PCAGGS-C31CNLS-pA transgene were crossed to heterozygous C31 reporter mice carrying the C31 reporter construct in the ROSA26 locus (SEQ ID NO:106) (FIG. 8). Offspring of the crosses were genotyped for the presence of the pCAGGS-C31CNLS-pA transgene by the PCR assay described in section C as well as for the ROSA26-C31 reporter allele by a LacZ-specific PCR assay. The PCR was performed using tail DNA and the primers β-Gal 3 (SEQ ID NO:102) and β-Gal 4 (SEQ ID NO:103) amplifying a fragment of 315 bp. The PCR reaction contained 5 μl PCR buffer (Invitrogen), 2.5 μl 50 mM MgCl₂, 2 μl 10 mM dNTP-mix, 1 μl (10 pmol) of each primer, 0.4 μl Taq-polymerase (5 U/μl) and water to a volume of 50 μl. The program used for the PCR reactions was: 94° C. for 1 min, 60° C. for 1 min and 72° C. for 1 min in 30 cycles.

Testes from mice carrying the pCAGGS-C31CNLS-pA transgene as well as the reporter locus and from a control mouse carrying the reporter allele only were dissected. The tissues were imbedded in OCT Tissue freezing medium (Leica/Jung) and frozen in liquid nitrogen. Cryosections were generated from the embedded tissues using a Leica CM3050 cryomicrotome, dried on polylysine-coated slides for 1-4 hours and then stained as follows: Sections were fixed in 0.2% glutaraldehyde, 5 mM EGTA, 2 mM MgCl₂ in 0.1 M PB (K₂HPO₄/KH₂PO₄, pH 7.3) for 5 min at room temperature and washed in wash buffer (2 mM MgCl₂, 0.02% Nonidet-40 in PB in 0.1 M PB) 3 times for 15 min. Then sections were stained in X-Gal-solution (0.6 mg/ml X-Gal in DMSO, 5 mM potassium hexacyanoferrat III, 5 mM potassium hexacyanoferrat II in LacZ wash buffer) overnigth at 37° C. After staining-sections were washed in 1×PBS twice for 5 min. Dehydration was performed by washing the sections first with 70%, 96% and 100% ethanol for 2 min each, then with a 1:1 mix of ethanol and xylol for 5 min and in the end only with xylol for 5 min. Before taking pictures sections were mounted in Entellan.

E. Results: To identify transgenic founder mice carrying the pCAGGS-C31CNLS-pA transgene, 29 mice born from the injection experiment were analyzed for the presence of the transgene. 5 founder mice (3 females and 2 males) were identified. To analyze the activity of the C31-Int(CNLS) recombinase in transgenic mice, 2 of the female founder mice were crossed to heterozygous C31 reporter mice carrying a C31 reporter construct in the ROSA26 locus (FIG. 8). From each of these crosses, one offspring carrying the pCAGGS-C31CNLS-pA transgene as well as the C31 reporter allele was sacrificed. In oder to determine whether pCAGGS-C31CNLS-pA transgenic mice are able to delete an attB/P flanked DNA sequence in the mouse germline, tissue sections from the testes of the sacrificed animals were prepared and stained for β-galactosidase activity with X-Gal. FIG. 9 shows the result of the staining experiment for one of these mice (A) as well as a control mouse carrying only the reporter allele, but lacking the pCAGGS-C31CNLS-pA transgene (B). Clear staining can be detected in the maturing sperm cells in about 50% of the tubules with the proportion of β-galactosidase expressing cells ranging between 10 and 100. No staining could be detected for the control mouse. This clearly demonstrates that C31-int-mediated recombination has taken place during spermatogenesis in the pCAGGS-C31CNLS-pA transgenic mice. These results show that the C31-int is functional in vivo, in a transgenic mouse system and therefore provides a new tool to introduce specific deletions, inversions or integrations into the mouse germline.

[image]

What is claimed is:

1. A fusion protein comprising
(a) a recombinase domain comprising a recombinase protein or a mutant thereof having a recombinase activity similar to that of the corresponding wild-type recombinase, and
(b) a signal peptide domain linked to said recombinase domain which directs nuclear import of said fusion protein in eucaryotic cells.

2. The fusion protein of claim 1, wherein the activity of the fusion protein in eucaryotic cells is significantly higher as compared to that of the wild-type recombinase corresponding to the recombinase of the recombinase domain.

3. The fusion protein of claim 1, wherein the recombinase domain comprises a recombinase protein belonging to the family of large serine recombinases or a mutant thereof having a recombinase activity similar to that of the corresponding wild-type recombinase.

4. The fusion protein of claim 3, wherein the recombinase protein is selected from the group consisting of bacteriophage φC31 integrase (C31-Int), coliphage P4 recombinase, Listeria phage recombinase, bacteriophage R4 Sre recombinase, CisA recombinase, XisF recombinase, transposon Tn4451 TnpX recombinase and lactococcal bacteriophage TP901-1 recombinase or a mutant thereof having a recombinase activity similar to that of the corresponding wild-type recombinase.

5. The fusion protein of claim 4, wherein the recombinase protein is a C31-Int protein or a mutant thereof having a recombinase activity similar to that of the corresponding wild-type C31-Int protein.

6. The fusion protein of claim 1, wherein the recombinase domain comprises a C31-Int protein having the amino acid sequence shown in SEQ ID NO:21.

7. The fusion protein of claim 5 comprising a C-terminal truncated form of said C31-Int protein.

8. The fusion protein of claim 7, wherein said truncated form of the C31-Int protein comprises amino acid residues 306 to 613 of SEQ ID NO:21.

9. The fusion protein according to claim 1 or 5, wherein the signal peptide domain is derived from a protein selected from the group consisting of yeast GAL4, yeast SKI3, yeast L29, yeast histone H2B, polyoma virus large T protein, VP1 capsid protein, VP2 capsid protein, SV40 VP1 capsid protein, VP2 capsid protein, adenovirus E1a, adenovirus DBP, influenza virus NS1, hepatitis virus core antigen, mammalian lamin, mammalian c-myc, mammalian max, mammalian c-myb, mammalian p53, mammalian c-erbA, mammalian jun, mammalian Tax, mammalian steroid receptor, mammalian Mx, and SV40 T-antigen.

10. The fusion protein of claim 9, wherein the signal peptide is derived from the SV40 T-antigen.

11. The fusion protein of claim 9, wherein the signal peptide domain has a length of 5 to 74 amino acid residues,

12. The fusion protein of claim 11, wherein the signal peptide domain has a length of 7 to 15 amino acid residues.

13. The fusion protein of claim 9, wherein the signal peptide domain comprises a segment of 6 amino acid residues having at least 2 positively charged basic amino acid residues.

14. The fusion protein of claim 13, wherein said basic amino acid residues are selected from lysine, arginine and histidine.

15. The fusion protein of claim 9, wherein the signal peptide domain comprises a sequence selected from the group consisting of any one of SEQ ID NOs:24 to 53.

16. The fusion protein of claim 9, wherein the signal peptide domain comprises the amino acid sequence Pro-Lys-Lys-Lys-Arg-Lys-Val (SEQ ID NO:53).

17. The fusion protein of claim 1, wherein the signal peptide domain is linked to the C-terminal of the recombinase domain.

18. The fusion protein of claim 1, wherein the signal peptide domain is linked to the recombinase domain through a linker peptide

19. The fusion protein of claim 18, wherein said linker has 1 to 30 essentially neutral amino acid residues.

20. The fusion protein of claim 1 comprising the amino acid sequence shown in SEQ ID NO:23.

21. A DNA coding for a fusion protein comprising
(a) a recombinase domain comprising a recombinase protein or a mutant thereof having a recombinase activity similar to that of the corresponding wild-type recombinase, and
(b) a signal peptide domain linked to said recombinase domain which directs nuclear import of said fusion protein in eucaryotic cells.

22. The DNA of claim 21, wherein the recombinase protein is a C31-Int protein or a mutant thereof having a recombinase activity similar to that of the corresponding wild-type C31-Int protein.

23. The DNA of claim 21 which codes for the amino acid sequence shown in SEQ ID NO:23.

24. A vector containing the DNA as defined in claim 21.

25. A microorganism containing the DNA of claim 21 or the vector of claim 24.

26. A process for preparing a fusion protein as defined in claim 1 which comprises culturing a microorganism as defined in claim 25 under conditions suitable for expression of said fusion protein and recovering said fusion protein.

27. A method for recombining a DNA molecule containing recognition sequences for a recombinase protein in a eucaryotic cell, said method comprising contacting the cell with a fusion protein according to claim 1 that recognizes said recognition sequences, wherein the fusion protein catalyzes recombination of the DNA molecule.

28. A cell containing a DNA sequence coding for a recombinase fusion protein in its genome, said recombinase fusion protein comprising
(a) a recombinase domain comprising a recombinase protein or a mutant thereof having a recombinase activity similar to that of the corresponding wild-type recombinase, and
(b) a signal peptide domain linked to said recombinase domain which directs nuclear import of said fusion protein in eucaryotic cells.

29. The cell of claim 28 which is a mammalian cell.

30. The cell of claim 28 also containing recognition sequences for the recombinase protein of the recombinase domain in its genome.

31. A transgenic organism containing a DNA sequence coding for a recombinase fusion protein in its genome, said recombinase fusion protein comprising
(a) a recombinase domain comprising a recombinase protein or a mutant thereof having a recombinase activity similar to that of the corresponding wild-type recombinase, and
(b) a signal peptide domain linked to said recombinase domain which directs nuclear import of said fusion protein in eucaryotic cells.

32. The transgenic organism of claim 31 which is a transgenic non-human mammal.

33. The transgenic organism of claim 31 also containing recognition sequences for the recombinase protein of the recombinase domain in its genome.

34. A method for recombining DNA molecules of cells or organisms containing recombinase recognition sequences for a recombinase protein of the recombinase domain of the fusion protein as defined in claim 1, which method comprises supplying the cells or organisms with a fusion protein as defined in claim 1 or with a DNA sequence of claim 21 or with a vector of claim 24 which are capable of expressing said fusion protein in the cell or organism.


SEQUENCE LISTING (108 sequences)

SEQ ID NO: 1 - DNA, 86 bp, Artificial Sequence
  Description of Artificial Sequence primer C31-4
cgtgacggtctcgaagccgcggtgcgggtgccagggcgtgcccttgggctccccgggcgcgtactccacctcacccatctggtcca

SEQ ID NO: 2 - DNA, 86 bp, Artificial Sequence
  Description of Artificial Sequence primer C31-5
cgtggaccagatgggtgaggtggagtacgcgcccggggagcccaagggcacgccctggcccacgcaccgcggcttcgagaccgtca

SEQ ID NO: 3 - DNA, 90 bp, Artificial Sequence
  Description of Artificial Sequence primer C31-6
gatccagaagcggttttcgggagtagtgccccaactggggtaacctttgagttctctcagttgggggcgtagggtcgccgacatgacacg

SEQ ID NO: 4 - DNA, 90 bp, Artificial Sequence
  Description of Artificial Sequence primer C31-7-2
gatccgtgtcatgtcggcgaccctacgcccccaactgagagaactcaaaggttaccccagttggggcactactcccgaaaaccgcttctg

SEQ ID NO: 5 - DNA, 7438 bp, Artificial Sequence
  Description of Artificial Sequence vector pRK64
cgtcatcaccgaaacgcgcgaggcagctgtggaatgtgtgtcagttagggtgtggaaagtccccaggctccccagcaggcagaagtatgcaaagcatgcatctcaattagtcagcaaccaggctccccagcaggcagaagtgtgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctaggaacagtcgacgacactgcagagacctacttcactaacaaccggtacagttcgtggaccagatgggtgaggtggagtacgcgcccggggagcccaagggcacgccctggcacccgcaccgcggcttcgagaccgtcacgaataacttcgtatagcatacattatacgaagttataagcttgcatgcctgcaggtcggccgccacgaccggccggccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgtgtcatgtcggcgaccctacgcccccaactgagagaactcaaaggttaccccagttggggcactactcccgaaaaccgcttctggatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgcagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcg

SEQ ID NO: 6 - DNA, 45 bp, Artificial Sequence
  Description of Artificial Sequence primer C31-1
ataagaatgcggccgcccgatatgacacaaggggttgtgaccggg

SEQ ID NO: 7 - DNA, 40 bp, Artificial Sequence
  Description of Artificial Sequence primer C31-3
ataagaatgcggccgcatccgccgctacgtcttccgtgcc

SEQ ID NO: 8 - DNA, 24 bp, Artificial Sequence
  Description of Artificial Sequence primer C31-8
cccgttggcaggaagcacttccgg

SEQ ID NO: 9 - DNA, 55 bp, Artificial Sequence
  Description of Artificial Sequence primer C31-9
ggatcctcgagccgcgggcggccgcctacgccgctacgtcttccgtgccgtcctg

SEQ ID NO: 10 - DNA, 5711 bp, Artificial Sequence
  Description of Artificial Sequence vector pCMV-C31-Int(wt)
aaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcccgatatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtaggcggcgcccgggctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttt

SEQ ID NO: 11 - DNA, 69 bp, Artificial Sequence
  Description of Artificial Sequence primer C31-2-2
tagaattccgctcgagagtctaaaccttcctcttcttcttaggcgccgctacgtcttccgtgccgtcct

SEQ ID NO: 12 - DNA, 5723 bp, Artificial Sequence
  Description of Artificial Sequence vector pCMV-C31-Int(CNLS)
cctgcaggtttaaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcccgatatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgcctaagaagaagaggaaggtttagactctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatg

SEQ ID NO: 13 - DNA, 4960 bp, Artificial Sequence
  Description of Artificial Sequence vector pCMV-Cre
aaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctcgaccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgcagatccactagttctagggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttt

SEQ ID NO: 14 - DNA, 3858 bp, Artificial Sequence
  Description of Artificial Sequence vector pRK50
aaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttt

SEQ ID NO: 15 - DNA, 6257 bp, Artificial Sequence
  Description of Artificial Sequence vector pRK64(deltaCre)
cgtcatcaccgaaacgcgcgaggcagctgtggaatgtgtgtcagttagggtgtggaaagtccccaggctccccagcaggcagaagtatgcaaagcatgcatctcaattagtcagcaaccaggctccccagcaggcagaagtgtgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctaggaacagtcgacgacactgcagagacctacttcactaacaaccggtacagttcgtggaccagatgggtgaggtggagtacgcgcccggggagcccaagggcacgccctggcacccgcaccgcggcttcgagaccgtcacgaatagatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgcagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcg

SEQ ID NO: 16 - DNA, 6252 bp, Artificial Sequence
  Description of Artificial Sequence vector pRK64(deltaInt)
cgtcatcaccgaaacgcgcgaggcagctgtggaatgtgtgtcagttagggtgtggaaagtccccaggctccccagcaggcagaagtatgcaaagcatgcatctcaattagtcagcaaccaggctccccagcaggcagaagtgtgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctaggaacagtcgacgacactgcagagacctacttcactaacaaccggtacagttcgtggaccagatgggtgaggtggagtacgcgcccggggagcccaaaggttaccccagttggggcactactcccgaaaaccgcttctggatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgcagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcg

SEQ ID NO: 17 - DNA, 25 bp, Artificial Sequence
  Description of Artificial Sequence primer P64-1
tcagcaaccaggctccccagcaggc

SEQ ID NO: 18 - DNA, 27 bp, Artificial Sequence
  Description of Artificial Sequence primer 64-4
gacgacagtatcggcctcaggaagatc

SEQ ID NO: 19 - DNA, 840 bp, Artificial Sequence
  “N” at position 536 may be adenine, cytosine, guanine and thymine; “Y” at position 537 may be thymine or cytosine: Description of Artificial Sequence oligonucleotide 80d
ggtaccgagctcggatcctctagtaacggccgccagtgtgctggaattcggcttcagcaaccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctaggaacagtcgacgacactgcagagacctacttcactaacaaccggtacagttcgtggaccagatgggtgaggtggagtacgcgcccggggagcccaaaggttaccccagttggggcactactcccgaaaaccgcttctggatccataacttcgtatagcatacattatacgaagttataccgggccaccatggtcgcgagtagcttggcactggggttgcttttgcgnygtcgtgactgggaaaaccctggcgttacccaacttaatcgccttgcagcacatccccctttcgccagctggcgtaatagcgaagaggcccgcaccgatcgcccttcccaacagttgcgcagctgaatggcgaatggcgctttgcctggcttccggcaccagaagcggtgccggaaagctggctggagtgcgatcttcctgaggccgatactgtcgtcaagccgaattctgcagatatccatcacactggcggccgctcgagcatgcatctagagggccaattcgccctatagtgagtcgtatt

SEQ ID NO: 20 - DNA, 1842 bp, Bacteriophage phi-C31
  CDS (1)..(1839)
atgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtag

SEQ ID NO: 21 - protein, 613 aa, Bacteriophage phi-C31
MTQGVVTGVDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 22 - DNA, 1863 bp, Artificial Sequence
  Description of Artificial Sequence DNA sequence coding for fusion protein C31-Int(CNLS)
atgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgcctaagaagaagaggaaggtttag

SEQ ID NO: 23 - protein, 620 aa, Artificial Sequence
  Description of Artificial Sequence Amino acid sequence for fusion protein C31-Int(CNLS)
MTQGVVTGVDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAAPKKKRKV

SEQ ID NO: 24 - protein, 43 aa, Artificial Sequence
  Description of Artificial Sequence NLS
MKKKKKKKKKKKCRLKKLKCSKEKPKCAKCLKKKKKRRRKTKR

SEQ ID NO: 25 - protein, 10 aa, Artificial Sequence
  Description of Artificial Sequence NLS
IKYFKKFPKD

SEQ ID NO: 26 - protein, 14 aa, Artificial Sequence
  Description of Artificial Sequence NLS
MTGSKTRKHRGSGA

SEQ ID NO: 27 - protein, 14 aa, Artificial Sequence
  Description of Artificial Sequence NLS
MTGSKHRKHPGSGA

SEQ ID NO: 28 - protein, 7 aa, Artificial Sequence
  Description of Artificial Sequence NLS
GKKRSKA

SEQ ID NO: 29 - protein, 14 aa, Artificial Sequence
  Description of Artificial Sequence NLS
PKKAREDVSRKRPR

SEQ ID NO: 30 - protein, 11 aa, Artificial Sequence
  Description of Artificial Sequence NLS
APKRKSGVSKC

SEQ ID NO: 31 - protein, 12 aa, Artificial Sequence
  Description of Artificial Sequence NLS
EEDGPQKKKRRL

SEQ ID NO: 32 - protein, 8 aa, Artificial Sequence
  Description of Artificial Sequence NLS
APTKRKGS

SEQ ID NO: 33 - protein, 7 aa, Artificial Sequence
  Description of Artificial Sequence NLS
PNKKKRK

SEQ ID NO: 34 - protein, 5 aa, Artificial Sequence
  Description of Artificial Sequence NLS
KRPRP

SEQ ID NO: 35 - protein, 11 aa, Artificial Sequence
  Description of Artificial Sequence NLS
CGGLSSKRPRP

SEQ ID NO: 36 - protein, 19 aa, Artificial Sequence
  Description of Artificial Sequence NLS
PPKKRMRRRIEPKKKKKRP

SEQ ID NO: 37 - protein, 11 aa, Artificial Sequence
  Description of Artificial Sequence NLS
PFLDRLRRDQK

SEQ ID NO: 38 - protein, 9 aa, Artificial Sequence
  Description of Artificial Sequence NLS
PKQKRKMAR

SEQ ID NO: 39 - protein, 9 aa, Artificial Sequence
  Description of Artificial Sequence NLS
SVTKKRKLE

SEQ ID NO: 40 - protein, 11 aa, Artificial Sequence
  Description of Artificial Sequence NLS
CGGAAKRVKLD

SEQ ID NO: 41 - protein, 9 aa, Artificial Sequence
  Description of Artificial Sequence NLS
PAAKRVKLD

SEQ ID NO: 42 - protein, 11 aa, Artificial Sequence
  Description of Artificial Sequence NLS
RQRRNELKRSP

SEQ ID NO: 43 - protein, 8 aa, Artificial Sequence
  Description of Artificial Sequence NLS
PQSRKKLR

SEQ ID NO: 44 - protein, 8 aa, Artificial Sequence
  Description of Artificial Sequence NLS
PLLKKIKQ

SEQ ID NO: 45 - protein, 7 aa, Artificial Sequence
  Description of Artificial Sequence NLS
PQPKKKP

SEQ ID NO: 46 - protein, 9 aa, Artificial Sequence
  Description of Artificial Sequence NLS
SKRVAKRKL

SEQ ID NO: 47 - protein, 9 aa, Artificial Sequence
  Description of Artificial Sequence NLS
ASKSRKRKL

SEQ ID NO: 48 - protein, 16 aa, Artificial Sequence
  Description of Artificial Sequence NLS
GGLCSARLHRHALLAT

SEQ ID NO: 49 - protein, 8 aa, Artificial Sequence
  Description of Artificial Sequence NLS
RKTKKKIK

SEQ ID NO: 50 - protein, 8 aa, Artificial Sequence
  Description of Artificial Sequence NLS
RKLKKLGN

SEQ ID NO: 51 - protein, 8 aa, Artificial Sequence
  Description of Artificial Sequence NLS
RKDRRGGR

SEQ ID NO: 52 - protein, 18 aa, Artificial Sequence
  Description of Artificial Sequence NLS
DTREKKKFLKRRLLRLDE

SEQ ID NO: 53 - protein, 7 aa, Artificial Sequence
  Description of Artificial Sequence NLS
PKKKRKV

SEQ ID NO: 54 - DNA, 1410 bp, Bacteriophage R4
  CDS (1)..(1407)
atgaatcgaggggggcccactgtacgggccgacatctacgtccgaatcagcctggaccgcacaggggaagagctcggggtcgagcgccaggaggagtcgtgtcgcgagctctgcaagagcctcggcatggaggtggggcaggtgtgggtcgacaacgacctgagcgccaccaagaagaacgtcgtccgccctgacttcgaggcgatgatcgcgagcaacccgcaggcgatcgtctgctggcacaccgaccggctcatccgcgtcacgcgggacctggagcgggtgatcgacctcggagtcaacgtccacgccgtgatggccggacacctggacctgtccaccccggccggccgagccgtcgcccgcacggtgacggcctgggccacgtacgagggcgagcagaaggctgagcgccagaagctcgccaacatccagaacgcccgcgccggcaagccgtacacccccggcatccgccccttcgggtacggcgacgaccacatgaccatcgtgacggccgaggcggacgccatccgcgacggcgcgaagatgatcctcgacggctggtccctgtcggccgtggctcgctactgggaggagctcaagctccagtcgccccggagtatggccgcaggcggcaagggctggtctctgcggggcgtaaagaaggtgctgacctccccgcgctacgtcgggcggtccagctacctcggggaggtcgtgggcgatgctcagtggccgcccatcctcgacccggacgtctactacggggtcgtggccatcctgaacaaccccgaccgcttcagcgggggccctcggaccggccgcacccccggcacgctgctcgcaggcatcgccttgtgcggtgagtgcggcaagacggtcagtggacgcggctaccgaggtgtcctggtctacggatgtaaggacacgcacactcggacgcctcggagcatcgctgacggccgcgcgagcagctcgaccctcgcccggctcatgttccccgacttcctgcccggcctcctggcctctgggcaggccgaggacggccagtcggcagcatccaagcactcggaggcccagacgctgcgcgagcgccttgacgggctggctacggcctacgcggagggtgcgatcagcctgtctcagatgacggccggctcggaagcactgcggaagaagctggaggtgatcgaagccgacctcgtgggctcggcaggcatcccgcccttcgatccagtggccggagtggctggcctgatctccggctggcccaccacgcctctcccgacgcgtcgagcatgggtggacttctgcctggtggtcacgctgaacacccagaaggggcgccatgcgtcgagcatgaccgtggacgaccacgtcaccatcgagtggcgagacgtggccgagtag

SEQ ID NO: 55 - protein, 469 aa, Bacteriophage R4
MNRGGPTVRADIYVRISLDRTGEELGVERQEESCRELCKSLGMEVGQVWVDNDLSATKKNVVRPDFEAMIASNPQAIVCWHTDRLIRVTRDLERVIDLGVNVHAVMAGHLDLSTPAGRAVARTVTAWATYEGEQKAERQKLANIQNARAGKPYTPGIRPFGYGDDHMTIVTAEADAIRDGAKMILDGWSLSAVARYWEELKLQSPRSMAAGGKGWSLRGVKKVLTSPRYVGRSSYLGEVVGDAQWPPILDPDVYYGVVAILNNPDRFSGGPRTGRTPGTLLAGIALCGECGKTVSGRGYRGVLVYGCKDTHTRTPRSIADGRASSSTLARLMFPDFLPGLLASGQAEDGQSAASKHSEAQTLRERLDGLATAYAEGAISLSQMTAGSEALRKKLEVIEADLVGSAGIPPFDPVAGVAGLISGWPTTPLPTRRAWVDFCLVVTLNTQKGRHASSMTVDDHVTIEWRDVAE

SEQ ID NO: 56 - DNA, 1503 bp, CisA recombinase
  CDS (1)..(1500)
gtgatagcaatatatgtaagggtatcgaccgaggaacaagcgatcaagggatcgagcatcgacagccaaatcgaggcctgtataaagaaagcagggactaaagatgtgctgaagtatgcagatgaaggattttcaggagagcttttagaacgtccggctttgaatcgcttgagggaggatgcaagcaagggacttataagtcaagtcatttgttacgatcctgaccgtctttctcggaaattaatgaatcagctaatcattgatgacgaattgcgaaagcgaaacatacctttgatttttgtaaatggtgaatacgccaattctccagaaggtcaattgtttttcgcaatgcgcggggcaatctcagaatttgaaaaagccaaaatcaaagaacggacatcaagcggccgacttcaaaaaatgaaaaaaggcatgatcattaaagattctaaactatatggctataaatttgttaaagagaaaagaactcttgagatattagaagaggaagcaaaaatcattcggatgatttttaactatttcaccgatcataaaagcccttttttcggcagagtaaatggtattgctctacatttaactcagatgggggttaaaacaaaaaaaggcgccaaagtatggcacaggcaggttgttcggcaaatattaatgaactcttcctataagggtgaacatagacagtataaatatgatacagagggttcctatgtttcaaagcaggcagggaacaaatctataattaaaataaggcctgaagaagaacaaatcactgtgacaattccagcaattgttccagctgaacaatgggattatgctcaagaactcttaggtcaaagtaaaagaaaacacttgagtatcagccctcacaattacttgttatcgggtttggttagatgcggaaaatgcggaaataccatgacagggaagaaaagaaaatcacatggtaaagactactatgtatatacttgccggaaaaattattctggcgcaaaggaccgcggctgcggaaaagaaatgtctgagaataaattgaaccggcatgtatggggtgaaatttttaaattcatcacaaatcctcaaaagtatgtttcttttaaagaggctgaacaatcaaatcacctgtctgatgaattagaacttattgaaaaagagatagagaaaacaaaaaaaggccgcaagcgtcttttaacgctaatcagcctaagcgatgacgatgatttagacatagatgaaatcaaagcacaaattattgaactgcaaaaaaagcaaaatcagcttactgaaaagtgtaacagaatccagtcaaaaatgaaagtcctagatgatacgagctcaagtgaaaatgctctaaaaagagccatcgactattttcaatcaatcggtgcagataacttaactcttgaagataaaaaaacaattgttaactttatcgtgaaagaagttaccattgtggattctgacaccatatatattgaaacgtattaa

SEQ ID NO: 57 - protein, 500 aa, CisA recombinase
VIAIYVRVSTEEQAIKGSSIDSQIEACIKKAGTKDVLKYADEGFSGELLERPALNRLREDASKGLISQVICYDPDRLSRKLMNQLIIDDELRKRNIPLIFVNGEYANSPEGQLFFAMRGAISEFEKAKIKERTSSGRLQKMKKGMIIKDSKLYGYKFVKEKRTLEILEEEAKIIRMIFNYFTDHKSPFFGRVNGIALHLTQMGVKTKKGAKVWHRQVVRQILMNSSYKGEHRQYKYDTEGSYVSKQAGNKSIIKIRPEEEQITVTIPAIVPAEQWDYAQELLGQSKRKHLSISPHNYLLSGLVRCGKCGNTMTGKKRKSHGKDYYVYTCRKNYSGAKDRGCGKEMSENKLNRHVWGEIFKFITNPQKYVSFKEAEQSNHLSDELELIEKEIEKTKKGRKRLLTLISLSDDDDLDIDEIKAQIIELQKKQNQLTEKCNRIQSKMKVLDDTSSSENALKRAIDYFQSIGADNLTLEDKKTIVNFIVKEVTIVDSDTIYIETY

SEQ ID NO: 58 - DNA, 1545 bp, XisF recombinase
  CDS (1)..(1542)
atggaaaattggggttacgcgagagtgagcggtgaggaacagcaaacagataaaggtgcgttgcgtaaacaaatagaacgcttgcgtaatgctggatgttcaaaagtgtactgggatattcaatcgcggacaactgaagtcagagaagggctacaacaattaattaatgacttaaagacatcttcaacaggtaaggtaaaatcactgcaatttacccgcattgatcgcatcggctcatcatcgcggttgttttattcattgttagaggtattacgttccaagggaattaaactgatagccttagatcaaggcgttgacccagacagccttggcggggaactaacaattgatatgttactggctgctgccaaatttgaggtaagaatggtgacggagaggttaaaaagcgaacgtcgtcatagggtgaaccaaggaaaaagtcaccgagttgccccattaggataccgcaaagataaagataaatatatacgcgatcgctcaccatgtgtttgcttactagaaggacgcagagaattaacggtgtctgacttagcccagtatatttttcacactttttttgagtgcggttccgttgctgctactgtgcgtaagctgcactcagattttggtatagaaacaaaagttctgaattggaacaagctagaaaaatcttcccggattgttggcgacgacgacttagataaaattgcatttacaccaaataaaactaaccaccccttgcgttatccctggtctgggctaagatggtcaatccctggtttaaaagcgttattagttaaccctgtttacgccgggggtttgccctttgatacttacgttaaatcaaaaggaaaacgcaagcattttgacgagtggaaagtaaaatggggaacccacgacgatgaggcaatcattacctgtgaggaacatgaaagaataaaacagatgattcgagacaatcgcaataatcgatgggctgcaagagaagaaaacgaagtaaacccattttctaatttacttaaatgtacccattgcggcggctcaatgacacgccacgccaaacgtgtagataagagtggacaagctatctattattatcagtgccgattgtataaagctggcaactgtagcaataaaaatatgatttcatccaaaatattagatatccaagtaatggatttattggcacaagaagccgaacgtttagcaaatttggtggaaacagatgagccgcttattgtagaagaacccccagaagtaaaaacgctgcgcgcatccctgaatagtctggaaacattgccagcaagttcagcaattgaacaaattaaaaatgacctcaaagaacagattgcgatcgcactaggagcaaccaataatgcttctaaacaatctctgattgccaaggaaagaattatacaagcttttgctcataaaagttactggcaaggactaaacgctcaagataaacgagcaatcctcaatggttgcgtaaaaaaaatctccgtagatggtaactttgttacagctattgagtatcgttactag

SEQ ID NO: 59 - protein, 514 aa, XisF recombinase
MENWGYARVSGEEQQTDKGALRKQIERLRNAGCSKVYWDIQSRTTEVREGLQQLINDLKTSSTGKVKSLQFTRIDRIGSSSRLFYSLLEVLRSKGIKLIALDQGVDPDSLGGELTIDMLLAAAKFEVRMVTERLKSERRHRVNQGKSHRVAPLGYRKDKDKYIRDRSPCVCLLEGRRELTVSDLAQYIFHTFFECGSVAATVRKLHSDFGIETKVLNWNKLEKSSRIVGDDDLDKIAFTPNKTNHPLRYPWSGLRWSIPGLKALLVNPVYAGGLPFDTYVKSKGKRKHFDEWKVKWGTHDDEAIITCEEHERIKQMIRDNRNNRWAAREENEVNPFSNLLKCTHCGGSMTRHAKRVDKSGQAIYYYQCRLYKAGNCSNKNMISSKILDIQVMDLLAQEAERLANLVETDEPLIVEEPPEVKTLRASLNSLETLPASSAIEQIKNDLKEQIAIALGATNNASKQSLIAKERIIQAFAHKSYWQGLNAQDKRAILNGCVKKISVDGNFVTAIEYRY

SEQ ID NO: 60 - DNA, 2124 bp, Transposon Tn4451
  CDS (1)..(2121)
atgtcaaggacttcaagaattacagcactttacgagcgtttgtcaagagatgatgaccttactggcgagagtaattctattaccaatcaaaagaaatacctcgaagattatgcccgtaggaatggttttgagaacattcgccattttaccgatgacggattttcgggtgtaaatttcaatcgccctggctttcaatctctgataaaagaagttgaagcaggaaatgtagaaaccttgattgttaaggatatgagccgattggggcgaaattatctgcaagtaggtttttatacggaagttctgtttccacagaaaaatgtccgtttccttgcaattaacaacagtattgacagtaacaacgcttcggataatgactttgctccgtttttgaatattatgaacgaatggtatgccaaagacacaagcaacaaaatcaaggctatattcgatgcccgtatgaaagacggaaagcgttgtagcggttctatcccttatgggtataaccgactgccgagcgacaaacaaacgcttgtggttgaccctgtggcttcggaagtggtaaagcgtatctttactcttgccaatgatggcaaaagtacaagggcaatcgcagaaatactgaccgaagaaaaagttttaacccctgcggcatacgcaaaggaataccaccccgaacagtacaacggcaacaagttcacaaacccttatctttgggcaatgtcaacgataagaaatattttaggcaggcaggaatatctcggtcacaccgttttgcgaaagtcggtaagcacaaatttcaaacttcacaagagaaaaagcacagacgaagaagaacagtatgtatttccgaatacacacgagcctatcatatcgcaggaactttgggacagcgttcaaaaacgcagaagcagagtaaatcgtgcctcggcttggggaacgcacagcaaccgtttaagcggatatttgtactgtgccgattgcggaagaagaatgactttgcagacacattacagcaaaaaagacggttctgtgcagtattcttaccgttgcggtgggtatgcaagcagagtgaacagttgtaccagtcattcgattagtaccgataatgttgaagccttgatattatcatctgtcaaacgcttttcaaggtttgttctgaatgatgaacaagcatttgctttggaactgcaatctctttggaatgaaaaacaggaggaaaagccgaaacacaatcaatcggaactgcaacgctgtcagaaacgctatgacgaactctctacccttgttcgtggcttgtatgaaaatcttatgtcgggattactgcccgaaagacagtataagcaactgatgaaacagtatgatgacgagcaggcagagttggaaacgaaaatggaaacgatgaaaacagaacttgccgaagaaaaagtaagttccgttgatattaagcatttcatttcgctgatacgcaagtgtaaaaatcctacggaaatctccgatacaatgtttaatgaacttgttgataagatagtggtttatgaagcagagggtgtgggaaaagcacgaacacaaaaggtcgatatttattttaactatgtcggtcaagtggatattgcctataccgaagaagaacttgccgagatagaaacacagaaagagcaggaggaacagcaacgcttggcaagacagcgcaagcgtgaaaaagcctaccgagaaaagcgaaaggcacagaaaatcgctgaaaacggtggcgaaatcgttaagacaaaggtttgccctcattgcaacaaagagtttatcccgacaagcaaccgacaggtgttctgttccaaagagtgctgctatcaagcaaggcaagacaaaaagaaaacagaccgagaagcagaacgaggaaatcactattaccgacagcgtgtatgtgctgtgtgcggcaattcctattggcctacacacagccaacagaaattctgctccgaagaatgtcaaagggtaaatcacaataagaaaacattggaattttaccaccataaaaaagaaaaggagaagctgcaatgcaaagatttatcacagacgaaagaacgggtatccgatatgaacttatcggggactattactacccctgcttaa

SEQ ID NO: 61 - protein, 707 aa, Transposon Tn4451
MSRTSRITALYERLSRDDDLTGESNSITNQKKYLEDYARRNGFENIRHFTDDGFSGVNFNRPGFQSLIKEVEAGNVETLIVKDMSRLGRNYLQVGFYTEVLFPQKNVRFLAINNSIDSNNASDNDFAPFLNIMNEWYAKDTSNKIKAIFDARMKDGKRCSGSIPYGYNRLPSDKQTLVVDPVASEVVKRIFTLANDGKSTRAIAEILTEEKVLTPAAYAKEYHPEQYNGNKFTNPYLWAMSTIRNILGRQEYLGHTVLRKSVSTNFKLHKRKSTDEEEQYVFPNTHEPIISQELWDSVQKRRSRVNRASAWGTHSNRLSGYLYCADCGRRMTLQTHYSKKDGSVQYSYRCGGYASRVNSCTSHSISTDNVEALILSSVKRFSRFVLNDEQAFALELQSLWNEKQEEKPKHNQSELQRCQKRYDELSTLVRGLYENLMSGLLPERQYKQLMKQYDDEQAELETKMETMKTELAEEKVSSVDIKHFISLIRKCKNPTEISDTMFNELVDKIVVYEAEGVGKARTQKVDIYFNYVGQVDIAYTEEELAEIETQKEQEEQQRLARQRKREKAYREKRKAQKIAENGGEIVKTKVCPHCNKEFIPTSNRQVFCSKECCYQARQDKKKTDREAERGNHYYRQRVCAVCGNSYWPTHSQQKFCSEECQRVNHNKKTLEFYHHKKEKEKLQCKDLSQTKERVSDMNLSGTITTPA

SEQ ID NO: 62 - DNA, 1420 bp, XisA recombinase
  CDS (1)..(1416)
atgcaaaatcagggtcaagacaaatatcaacaagcctttgcagacttagagccactttcatctaccgacggcagttttctcggctcaagtctgcaagcacagcagcaaagagaacacatgagaacaaaagtactacaagacctagacaaggtaaatctgcgtttgaagtctgcaaagacgaaagtctcagttcgagaatctaacggaagtctgcaattacgagcaacgttaccaattaaacctggagataaggacaccaacggtacaggcagaaagcaatacaatctcagcttgaatatccctgcaaacttggatggactgaagacggctgaggaagaagcttatgaattaggtaaattaatcgctcggaaaacctttgaatggaatgataaatatttaggcaaagaagccactaaaaaagattcacaaacaataggtgatttactagaaaaatttgcagaagagtattttaaaacccataaacgcaccactaaaagcgaacatacctttttttactatttttcccgcacccaacgatataccaattccaaagatttagcaacggcggaaaatctcatcaattcaattgagcaaatcgataaagaatgggcgagatataatgccgccagagccatatcagctttttgcataacattcaatatagaaattgatttgtcccagtattccaaaatgcctgatcgcaattcgcgcaacatccccacagatgcagaaatactatcaggaattaccaaatttgaagactatctagttaccagaggaaatcaagttaatgaagatgtaaaagatagctggcaactttggcgctggacatatggaatgttagcagtttttggtttacgccccagggaaatttttattaaccctaatattgattggtggttaagcaaagagaatatagacctcacatggaaagtagacaaagaatgtaaaactggtgaaagacaagcattacccttacataaagaatggattgatgagtttgatttaagaaatccgaaatatttagaaatgctggcaacagcaattagtaaaaaagataaaacaaatcatgctgaaataacagccttaactcagcgtattagttggtggtttcggaaagtcgaattagattttaaaccctatgatttacgtcacgcctgggcaatcagagcgcatattttaggcataccaatcaaagcggcggctgataatttggggcatagtatgcaggttcatacacaaacctatcagcgctggttctcgctagatatgcggaagttagcgattaatcaggctttgactaagaggaatgaatttgaggtgattagggaggagaatgctaaattgcagatagaaaatgaaaggttgaggatggaaattgagaagttaaagatggaaatagcttataagaatagttgag

SEQ ID NO: 63 - protein, 472 aa, XisA recombinase
MQNQGQDKYQQAFADLEPLSSTDGSFLGSSLQAQQQREHMRTKVLQDLDKVNLRLKSAKTKVSVRESNGSLQLRATLPIKPGDKDTNGTGRKQYNLSLNIPANLDGLKTAEEEAYELGKLIARKTFEWNDKYLGKEATKKDSQTIGDLLEKFAEEYFKTHKRTTKSEHTFFYYFSRTQRYTNSKDLATAENLINSIEQIDKEWARYNAARAISAFCITFNIEIDLSQYSKMPDRNSRNIPTDAEILSGITKFEDYLVTRGNQVNEDVKDSWQLWRWTYGMLAVFGLRPREIFINPNIDWWLSKENIDLTWKVDKECKTGERQALPLHKEWIDEFDLRNPKYLEMLATAISKKDKTNHAEITALTQRISWWFRKVELDFKPYDLRHAWAIRAHILGIPIKAAADNLGHSMQVHTQTYQRWFSLDMRKLAINQALTKRNEFEVIREENAKLQIENERLRMEIEKLKMEIAYKNS

SEQ ID NO: 64 - DNA, 1008 bp, Artificial Sequence
  CDS (1)..(1005)
  Description of Artificial Sequence vector pBS-SSV3
atgacgaaagataagacgcgttataaatacggggattatattttacgcgagaggaaagggcggtattatgtttacaagctagagtatgaaaacggtgaggtaaaagagcgttacgtgggtcctttagctgacgtcgttgaatcatatctaaaaatgaaattaggggtcgtaggggatactcccctacaagcggatccccccggtttcgagcccgggacaagcggaagcggtggtggaaaagagggaactgaacgacgtaaaatagcgttggttgccaatttgcgccaatacgcgacggacggcaacataaaggcgttctacaactatctcatgaacgaaagggggataagcgaaaaaactgcaaaggactacatcaatgctatatcaaagccgtataaagagacgagagacgcacagaaggcttaccgactctttgcacgtttcttagcgtcacgcaatatcatacatgatgaatttgcggataaaatattgaaagcggtaaaggtgaagaaggcgaacgctgatatctacattccaacgttggaagagataaaaaggacgttacaattagcaaaagactatagcgaaaacgtctacttcatctaccgtatcgctctcgagtcgggcgttaggctgagcgaaatactgaaagtgctgaaggaacccgaaagggacatttgcggtaacgacgtctgttattatccgcttagttggactaggggatataagggcgtcttctatgtattccacataacgcctctgaagagagtagaggtgacgaagtgggcaatagcggactttgaacgacgtcataaggacgctatagcgataaagtacttccgcaaattcgtagcgtctaagatggctgagctaagcgtaccgttagatattatcgattttattcaagggcgtaaaccgacacgcgttttaacgcaacattacgtatcgctcttcggcatagcgaaagagcaatataaaaagtatgcggaatggctaaaaggggtctga

SEQ ID NO: 65 - protein, 335 aa, Artificial Sequence
  Description of Artificial Sequence vector pBS-SSV3
MTKDKTRYKYGDYILRERKGRYYVYKLEYENGEVKERYVGPLADVVESYLKMKLGVVGDTPLQADPPGFEPGTSGSGGGKEGTERRKIALVANLRQYATDGNIKAFYNYLMNERGISEKTAKDYINAISKPYKETRDAQKAYRLFARFLASRNIIHDEFADKILKAVKVKKANADIYIPTLEEIKRTLQLAKDYSENVYFIYRIALESGVRLSEILKVLKEPERDICGNDVCYYPLSWTRGYKGVFYVFHITPLKRVEVTKWAIADFERRHKDAIAIKYFRKFVASKMAELSVPLDIIDFIQGRKPTRVLTQHYVSLFGIAKEQYKKYAEWLKGV

SEQ ID NO: 66 - DNA, 1441 bp, Artificial Sequence
  Description of Artificial Sequence DNA sequence coding for fusion protein NLS-XisA
atgcccaagaagaagaggaaggtgcaaaatcagggtcaagacaaatatcaacaagcctttgcagacttagagccactttcatctaccgacggcagttttctcggctcaagtctgcaagcacagcagcaaagagaacacatgagaacaaaagtactacaagacctagacaaggtaaatctgcgtttgaagtctgcaaagacgaaagtctcagttcgagaatctaacggaagtctgcaattacgagcaacgttaccaattaaacctggagataaggacaccaacggtacaggcagaaagcaatacaatctcagcttgaatatccctgcaaacttggatggactgaagacggctgaggaagaagcttatgaattaggtaaattaatcgctcggaaaacctttgaatggaatgataaatatttaggcaaagaagccactaaaaaagattcacaaacaataggtgatttactagaaaaatttgcagaagagtattttaaaacccataaacgcaccactaaaagcgaacatacctttttttactatttttcccgcacccaacgatataccaattccaaagatttagcaacggcggaaaatctcatcaattcaattgagcaaatcgataaagaatgggcgagatataatgccgccagagccatatcagctttttgcataacattcaatatagaaattgatttgtcccagtattccaaaatgcctgatcgcaattcgcgcaacatccccacagatgcagaaatactatcaggaattaccaaatttgaagactatctagttaccagaggaaatcaagttaatgaagatgtaaaagatagctggcaactttggcgctggacatatggaatgttagcagtttttggtttacgccccagggaaatttttattaaccctaatattgattggtggttaagcaaagagaatatagacctcacatggaaagtagacaaagaatgtaaaactggtgaaagacaagcattacccttacataaagaatggattgatgagtttgatttaagaaatccgaaatatttagaaatgctggcaacagcaattagtaaaaaagataaaacaaatcatgctgaaataacagccttaactcagcgtattagttggtggtttcggaaagtcgaattagattttaaaccctatgatttacgtcacgcctgggcaatcagagcgcatattttaggcataccaatcaaagcggcggctgataatttggggcatagtatgcaggttcatacacaaacctatcagcgctggttctcgctagatatgcggaagttagcgattaatcaggctttgactaagaggaatgaatttgaggtgattagggaggagaatgctaaattgcagatagaaaatgaaaggttgaggatggaaattgagaagttaaagatggaaatagcttataagaatagttgag

SEQ ID NO: 67 - protein, 479 aa, Artificial Sequence
  Description of Artificial Sequence Amino acid sequence for fusion protein NLS-XisA
MPKKKRKVQNQGQDKYQQAFADLEPLSSTDGSFLGSSLQAQQQREHMRTKVLQDLDKVNLRLKSAKTKVSVRESNGSLQLRATLPIKPGDKDTNGTGRKQYNLSLNIPANLDGLKTAEEEAYELGKLIARKTFEWNDKYLGKEATKKDSQTIGDLLEKFAEEYFKTHKRTTKSEHTFFYYFSRTQRYTNSKDLATAENLINSIEQIDKEWARYNAARAISAFCITFNIEIDLSQYSKMPDRNSRNIPTDAEILSGITKFEDYLVTRGNQVNEDVKDSWQLWRWTYGMLAVFGLRPREIFINPNIDWWLSKENIDLTWKVDKECKTGERQALPLHKEWIDEFDLRNPKYLEMLATAISKKDKTNHAEITALTQRISWWFRKVELDFKPYDLRHAWAIRAHILGIPIKAAADNLGHSMQVHTQTYQRWFSLDMRKLAINQALTKRNEFEVIREENAKLQIENERLRMEIEKLKMEIAYKNS

SEQ ID NO: 68 - DNA, 1029 bp, Artificial Sequence
  Description of Artificial Sequence DNA sequence coding for fusion protein NLS-Ssv
atgcccaagaagaagaggaaggtgacgaaagataagacgcgttataaatacggggattatattttacgcgagaggaaagggcggtattatgtttacaagctagagtatgaaaacggtgaggtaaaagagcgttacgtgggtcctttagctgacgtcgttgaatcatatctaaaaatgaaattaggggtcgtaggggatactcccctacaagcggatccccccggtttcgagcccgggacaagcggaagcggtggtggaaaagagggaactgaacgacgtaaaatagcgttggttgccaatttgcgccaatacgcgacggacggcaacataaaggcgttctacaactatctcatgaacgaaagggggataagcgaaaaaactgcaaaggactacatcaatgctatatcaaagccgtataaagagacgagagacgcacagaaggcttaccgactctttgcacgtttcttagcgtcacgcaatatcatacatgatgaatttgcggataaaatattgaaagcggtaaaggtgaagaaggcgaacgctgatatctacattccaacgttggaagagataaaaaggacgttacaattagcaaaagactatagcgaaaacgtctacttcatctaccgtatcgctctcgagtcgggcgttaggctgagcgaaatactgaaagtgctgaaggaacccgaaagggacatttgcggtaacgacgtctgttattatccgcttagttggactaggggatataagggcgtcttctatgtattccacataacgcctctgaagagagtagaggtgacgaagtgggcaatagcggactttgaacgacgtcataaggacgctatagcgataaagtacttccgcaaattcgtagcgtctaagatggctgagctaagcgtaccgttagatattatcgattttattcaagggcgtaaaccgacacgcgttttaacgcaacattacgtatcgctcttcggcatagcgaaagagcaatataaaaagtatgcggaatggctaaaaggggtctga

SEQ ID NO: 69 - protein, 342 aa, Artificial Sequence
  Description of Artificial Sequence Amino acid sequence for fusion protein NLS-Ssv
MPKKKRKVTKDKTRYKYGDYILRERKGRYYVYKLEYENGEVKERYVGPLADVVESYLKMKLGVVGDTPLQADPPGFEPGTSGSGGGKEGTERRKIALVANLRQYATDGNIKAFYNYLMNERGISEKTAKDYINAISKPYKETRDAQKAYRLFARFLASRNIIHDEFADKILKAVKVKKANADIYIPTLEEIKRTLQLAKDYSENVYFIYRIALESGVRLSEILKVLKEPERDICGNDVCYYPLSWTRGYKGVFYVFHITPLKRVEVTKWAIADFERRHKDAIAIKYFRKFVASKMAELSVPLDIIDFIQGRKPTRVLTQHYVSLFGIAKEQYKKYAEWLKGV

SEQ ID NO: 70 - DNA, 3908 bp, Artificial Sequence
  Description of Artificial Sequence vector pBS-SSV3
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgcccgatatgacgaaagataagacgcgttataaatacggggattatattttacgcgagaggaaagggcggtattatgtttacaagctagagtatgaaaacggtgaggtaaaagagcgttacgtgggtcctttagctgacgtcgttgaatcatatctaaaaatgaaattaggggtcgtaggggatactcccctacaagcggatccccccggtttcgagcccgggacaagcggaagcggtggtggaaaagagggaactgaacgacgtaaaatagcgttggttgccaatttgcgccaatacgcgacggacggcaacataaaggcgttctacaactatctcatgaacgaaagggggataagcgaaaaaactgcaaaggactacatcaatgctatatcaaagccgtataaagagacgagagacgcacagaaggcttaccgactctttgcacgtttcttagcgtcacgcaatatcatacatgatgaatttgcggataaaatattgaaagcggtaaaggtgaagaaggcgaacgctgatatctacattccaacgttggaagagataaaaaggacgttacaattagcaaaagactatagcgaaaacgtctacttcatctaccgtatcgctctcgagtcgggcgttaggctgagcgaaatactgaaagtgctgaaggaacccgaaagggacatttgcggtaacgacgtctgttattatccgcttagttggactaggggatataagggcgtcttctatgtattccacataacgcctctgaagagagtagaggtgacgaagtgggcaatagcggactttgaacgacgtcataaggacgctatagcgataaagtacttccgcaaattcgtagcgtctaagatggctgagctaagcgtaccgttagatattatcgattttattcaagggcgtaaaccgacacgcgttttaacgcaacattacgtatcgctcttcggcatagcgaaagagcaatataaaaagtatgcggaatggctaaaaggggtctgactcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 71 - DNA, 3927 bp, Artificial Sequence
  Description of Artificial Sequence vector pBS-SSV4
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgcaccatgcccaagaagaagaggaaggtgacgaaagataagacgcgttataaatacggggattatattttacgcgagaggaaagggcggtattatgtttacaagctagagtatgaaaacggtgaggtaaaagagcgttacgtgggtcctttagctgacgtcgttgaatcatatctaaaaatgaaattaggggtcgtaggggatactcccctacaagcggatccccccggtttcgagcccgggacaagcggaagcggtggtggaaaagagggaactgaacgacgtaaaatagcgttggttgccaatttgcgccaatacgcgacggacggcaacataaaggcgttctacaactatctcatgaacgaaagggggataagcgaaaaaactgcaaaggactacatcaatgctatatcaaagccgtataaagagacgagagacgcacagaaggcttaccgactctttgcacgtttcttagcgtcacgcaatatcatacatgatgaatttgcggataaaatattgaaagcggtaaaggtgaagaaggcgaacgctgatatctacattccaacgttggaagagataaaaaggacgttacaattagcaaaagactatagcgaaaacgtctacttcatctaccgtatcgctctcgagtcgggcgttaggctgagcgaaatactgaaagtgctgaaggaacccgaaagggacatttgcggtaacgacgtctgttattatccgcttagttggactaggggatataagggcgtcttctatgtattccacataacgcctctgaagagagtagaggtgacgaagtgggcaatagcggactttgaacgacgtcataaggacgctatagcgataaagtacttccgcaaattcgtagcgtctaagatggctgagctaagcgtaccgttagatattatcgattttattcaagggcgtaaaccgacacgcgttttaacgcaacattacgtatcgctcttcggcatagcgaaagagcaatataaaaagtatgcggaatggctaaaaggggtctgactcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 72 - DNA, 3351 bp, Artificial Sequence
  Description of Artificial Sequence vector pBS-SSVs
cgacctcgagtcagaccccttttagccattccgcatactttttatattgctctttcgctatgccgaagagcgatacgtaatgttgcgttaaaacgcgtgtcggtttacgcccttgaataaaatcgataatatctaacggtacgcttagctcagccatcttagacgctacgaatttgcggaagtactttatcgctatagcgtccttatgacgtcgttcaaagtccgctattgcccacttcgtcacctctactctcttcagaggcgttatgtggaatacatagaagacgcccttatatcccctagtccaactaagcggataataacagacgtcgttaccgcaaatgtccctttcgggttccttcagcactttcagtatttcgctcagcctaacgcccgactcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgatatcaagcttatcgataccgt

SEQ ID NO: 73 - DNA, 5730 bp, Artificial Sequence
  Description of Artificial Sequence vector pCMVC31(NNLS)
aaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcaccatgcccaagaagaagaggaaggtgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtaggcggcgcccgggctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttt

SEQ ID NO: 74 - DNA, 4886 bp, Artificial Sequence
  Description of Artificial Sequence vector pCMV-SSV
aaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcccgatatgacgaaagataagacgcgttataaatacggggattatattttacgcgagaggaaagggcggtattatgtttacaagctagagtatgaaaacggtgaggtaaaagagcgttacgtgggtcctttagctgacgtcgttgaatcatatctaaaaatgaaattaggggtcgtaggggatactcccctacaagcggatccccccggtttcgagcccgggacaagcggaagcggtggtggaaaagagggaactgaacgacgtaaaatagcgttggttgccaatttgcgccaatacgcgacggacggcaacataaaggcgttctacaactatctcatgaacgaaagggggataagcgaaaaaactgcaaaggactacatcaatgctatatcaaagccgtataaagagacgagagacgcacagaaggcttaccgactctttgcacgtttcttagcgtcacgcaatatcatacatgatgaatttgcggataaaatattgaaagcggtaaaggtgaagaaggcgaacgctgatatctacattccaacgttggaagagataaaaaggacgttacaattagcaaaagactatagcgaaaacgtctacttcatctaccgtatcgctctcgagtcgggcgttaggctgagcgaaatactgaaagtgctgaaggaacccgaaagggacatttgcggtaacgacgtctgttattatccgcttagttggactaggggatataagggcgtcttctatgtattccacataacgcctctgaagagagtagaggtgacgaagtgggcaatagcggactttgaacgacgtcataaggacgctatagcgataaagtacttccgcaaattcgtagcgtctaagatggctgagctaagcgtaccgttagatattatcgattttattcaagggcgtaaaccgacacgcgttttaacgcaacattacgtatcgctcttcggcatagcgaaagagcaatataaaaagtatgcggaatggctaaaaggggtctgactcgagggggggcccgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttt

SEQ ID NO: 75 - DNA, 4905 bp, Artificial Sequence
  Description of Artificial Sequence vector pCMV-SSV(NNLS)
aaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcaccatgcccaagaagaagaggaaggtgacgaaagataagacgcgttataaatacggggattatattttacgcgagaggaaagggcggtattatgtttacaagctagagtatgaaaacggtgaggtaaaagagcgttacgtgggtcctttagctgacgtcgttgaatcatatctaaaaatgaaattaggggtcgtaggggatactcccctacaagcggatccccccggtttcgagcccgggacaagcggaagcggtggtggaaaagagggaactgaacgacgtaaaatagcgttggttgccaatttgcgccaatacgcgacggacggcaacataaaggcgttctacaactatctcatgaacgaaagggggataagcgaaaaaactgcaaaggactacatcaatgctatatcaaagccgtataaagagacgagagacgcacagaaggcttaccgactctttgcacgtttcttagcgtcacgcaatatcatacatgatgaatttgcggataaaatattgaaagcggtaaaggtgaagaaggcgaacgctgatatctacattccaacgttggaagagataaaaaggacgttacaattagcaaaagactatagcgaaaacgtctacttcatctaccgtatcgctctcgagtcgggcgttaggctgagcgaaatactgaaagtgctgaaggaacccgaaagggacatttgcggtaacgacgtctgttattatccgcttagttggactaggggatataagggcgtcttctatgtattccacataacgcctctgaagagagtagaggtgacgaagtgggcaatagcggactttgaacgacgtcataaggacgctatagcgataaagtacttccgcaaattcgtagcgtctaagatggctgagctaagcgtaccgttagatattatcgattttattcaagggcgtaaaccgacacgcgttttaacgcaacattacgtatcgctcttcggcatagcgaaagagcaatataaaaagtatgcggaatggctaaaaggggtctgactcgagggggggcccgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttt

SEQ ID NO: 76 - DNA, 5290 bp, Artificial Sequence
  Description of Artificial Sequence vector pCMVXisA
agtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcccgatatgcaaaatcagggtcaagacaaatatcaacaagcctttgcagacttagagccactctcatctaccgacggcagttttctcggctcaagtctgcaagcacagcagcaaagagaacacatgagaacaaaagtactacaagacctagacaaggtaaatctgcgtttgaagtctgcaaagacgaaagtctcagttcgagaatctaacggaagtctgcaattacgagcaacgttaccaattaaacctggagataaggacaccaacggtacaggcagaaagcaatacaatctcagcttgaatatccctgcaaacttggatggactgaagacggctgaggaagaagcttatgaattaggtaaattaatcgctcggaaaacctttgaatggaatgataaatatttaggcaaagaagccactaaaaaagattcacaaacaataggtgatttactagaaaaatttgcagaagagtattttaaaacccataaacgcaccactaaaagcgaacatacctttttttactatttttcccgcacccaacgatataccaattccaaagatttagcaacggcggaaaatctcatcaattcaattgagcaaatcgataaagaatgggcgagatataatgccgccagagccatatcagctttttgcataacattcaatatagaaattgatttgtcccagtattccaaaatgcctgatcgcaattcgcgcaacatccccacagatgcagaaatactatcaggaattaccaaatttgaagactatctagttaccagaggaaatcaagttaatgaagatgtaaaagatagctggcaactttggcgctggacatatggaatgttagcagtttttggtttacgccccagggaaatttttattaaccctaatattgattggtggttaagcaaagagaatatagacctcacatggaaagtagacaaagaatgtaaaactggtgaaagacaagcattacccttacataaagaatggattgatgagtttgatttaagaaatccgaaatatttagaaatgctggcaacagcaattagtaaaaaagataaaacaaatcatgctgaaataacagccttaactcagcgtattagttggtggtttcggaaagtcgaattagattttaaaccctatgatttacgtcacgcctgggcaattagagcgcatattttaggcataccaatcaaagcggcggctgataatttggggcatagtatgcaggttcatacacaaacctatcagcgctggttctcgctagatatgcggaagttagcgattaatcaggctttgactaagaggaatgaatttgaggtgattagggaggagaatgctaaattgcagatagaaaatgaaaggttgaggatggaaattgagaagttaaagatggaaatagcttataagaatagttgagcggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggtttaaac

SEQ ID NO: 77 - DNA, 5309 bp, Artificial Sequence
  Description of Artificial Sequence vector pCMVXisANNLS
aaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcaccatgcccaagaagaagaggaaggtgcaaaatcagggtcaagacaaatatcaacaagcctttgcagacttagagccactttcatctaccgacggcagttttctcggctcaagtctgcaagcacagcagcaaagagaacacatgagaacaaaagtactacaagacctagacaaggtaaatctgcgtttgaagtctgcaaagacgaaagtctcagttcgagaatctaacggaagtctgcaattacgagcaacgttaccaattaaacctggagataaggacaccaacggtacaggcagaaagcaatacaatctcagcttgaatatccctgcaaacttggatggactgaagacggctgaggaagaagcttatgaattaggtaaattaatcgctcggaaaacctttgaatggaatgataaatatttaggcaaagaagccactaaaaaagattcacaaacaataggtgatttactagaaaaatttgcagaagagtattttaaaacccataaacgcaccactaaaagcgaacatacctttttttactatttttcccgcacccaacgatataccaattccaaagatttagcaacggcggaaaatctcatcaattcaattgagcaaatcgataaagaatgggcgagatataatgccgccagagccatatcagctttttgcataacattcaatatagaaattgatttgtcccagtattccaaaatgcctgatcgcaattcgcgcaacatccccacagatgcagaaatactatcaggaattaccaaatttgaagactatctagttaccagaggaaatcaagttaatgaagatgtaaaagatagctggcaactttggcgctggacatatggaatgttagcagtttttggtttacgccccagggaaatttttattaaccctaatattgattggtggttaagcaaagagaatatagacctcacatggaaagtagacaaagaatgtaaaactggtgaaagacaagcattacccttacataaagaatggattgatgagtttgatttaagaaatccgaaatatttagaaatgctggcaacagcaattagtaaaaaagataaaacaaatcatgctgaaataacagccttaactcagcgtattagttggtggtttcggaaagtcgaattagattttaaaccctatgatttacgtcacgcctgggcaatcagagcgcatattttaggcataccaatcaaagcggcggctgataatttggggcatagtatgcaggttcatacacaaacctatcagcgctggttctcgctagatatgcggaagttagcgattaatcaggctttgactaagaggaatgaatttgaggtgattagggaggagaatgctaaattgcagatagaaaatgaaaggttgaggatggaaattgagaagttaaagatggaaatagcttataagaatagttgagcggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttt

SEQ ID NO: 78 - DNA, 7608 bp, Artificial Sequence
  Description of Artificial Sequence vector pPGKnifD
tcgaggaattctaccgggtaggggaggcgcttttcccaaggcagtctggagcatgcgctttagcagccccgctgggcacttggcgctacacaagtggcctctggctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccggtacagttcgaatggctcttcccttccgtcaaatgcactcttgggattactccgaacctagcgatggggtgcaaatgtcagatcagataagttcgaataacttcgtatagcatacattatacgaagttataagcttgcatgcctgcaggtcggccgccacgaccggccggccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccagctgttgaaagctattaaaccacaaaaaggattactccggcccttatcacggttacgacggatttggatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgcagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcg

SEQ ID NO: 79 - DNA, 7523 bp, Artificial Sequence
  Description of Artificial Sequence vector pPGKnifD3′
tcgaggaattctaccgggtaggggaggcgcttttcccaaggcagtctggagcatgcgctttagcagccccgctgggcacttggcgctacacaagtggcctctggctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccggtacagttcgaataacttcgtatagcatacattatacgaagttataagcttgcatgcctgcaggtcggccgccacgaccggccggccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccagctgttgaaagctattaaaccacaaaaaggattactccggcccttatcacggttacgacggatttggatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgcagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcg

SEQ ID NO: 80 - DNA, 4754 bp, Artificial Sequence
  Description of Artificial Sequence vector pRK55pbsC31-Re
ggccgcccgatatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtaggcggcgcccgggctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggc

SEQ ID NO: 81 - DNA, 4773 bp, Artificial Sequence
  Description of Artificial Sequence vector pRK63pbsC31NLS-Re
ggccgcaccatgcccaagaagaagaggaaggtgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtaggcggcgcccgggctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggc

SEQ ID NO: 82 - DNA, 7803 bp, Artificial Sequence
  Description of Artificial Sequence vector pPGKattA1
tatcatgtctggatccgcgttaacacctaagaaggcgaagttttccttacaccttgcagatataaagtgtctaacagtttaaaatatccgatgaggcatatttatgttggacccgtagctcagccaggatagagcactggcctccggagccggaggtcccgggttcaaatcccggcgggtccgtatattactttttgattcagattagatttgtaaatctttattacaaggataatttgatcttgtatattggtaactctctactctataatttttatgagaaattcacagtcgtccctttataccataaatagctaagtttgtcaaagttcttattaaactctccatgtagagattaaatcggatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgcagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcgtcgaggaattctaccgggtaggggaggcgcttttcccaaggcagtctggagcatgcgctttagcagccccgctgggcacttggcgctacacaagtggcctctggctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccggtacagttcgaataacttcgtatagcatacattatacgaagttataagcttgcatgcctgcaggtcggccgccacgaccggccggccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatct

SEQ ID NO: 83 - DNA, 8167 bp, Artificial Sequence
  Description of Artificial Sequence vector pPGKattA2
tatcatgtctggatccgcgttaacacctaagaaggcgaagttttccttacaccttgcagatataaagtgtctaacagtttaaaatatccgatgaggcatatttatgttggacccgtagctcagccaggatagagcactggcctccggagccggaggtcccgggttcaaatcccggcgggtccgtatattactttttgattcagattagatttgtaaatctttattacaaggataatttgatcttgtatattggtaactctctactctataatttttatgagaaattcacagtcgtccctttataccataaatagctaagtttgtcaaagttcttattaaactctccatgtagagattaaatcggatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgcagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcgtcgaggaattctaccgggtaggggaggcgcttttcccaaggcagtctggagcatgcgctttagcagccccgctgggcacttggcgctacacaagtggcctctggctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccggtacagttcgaaggatccgcgttaacacctaagaaggcgaagttttccttacaccttgcagatataaagtgtctaacagtttaaaatatccgatgaggcatatttatgttggacccgtagctcagccaggatagagcactggcctccggagccggaggtcccgggttcaaatcccggcgggtccgtatattactttttgattcagattagatttgtaaatctttattacaaggataatttgatcttgtatattggtaactctctactctataatttttatgagaaattcacagtcgtccctttataccataaatagctaagtttgtcaaagttcttattaaactctccatgtagagattaaatcggatccttcgaataacttcgtatagcatacattatacgaagttataagcttgcatgcctgcaggtcggccgccacgaccggccggccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatct

SEQ ID NO: 84 - DNA, 51 bp, Artificial Sequence
  Description of Artificial Sequence primer XisA1
ataagaatgcggccgcccgatatgcaaaatcagggtcaagacaaatatcaa

SEQ ID NO: 85 - DNA, 76 bp, Artificial Sequence
  Description of Artificial Sequence primer XisA2
ataagaatgcggccgcaccatgcccaagaagaagaggaaggtgcaaaatcagggtcaagacaaatatcaacaagcc

SEQ ID NO: 86 - DNA, 44 bp, Artificial Sequence
  Description of Artificial Sequence primer XisA3
ataagaatgcggccgctcaactattcttataagctatttccatc

SEQ ID NO: 87 - DNA, 82 bp, Artificial Sequence
  Description of Artificial Sequence primer nifD1
cgatggctcttcccttccgtcaaatgcactcttgggattactccgaacctagcgatggggtgcaaatgtcagatcagataag

SEQ ID NO: 88 - DNA, 82 bp, Artificial Sequence
  Description of Artificial Sequence primer nifD2
cgcttatctgatctgacatttgcaccccatcgctaggttcggagtaatcccaagagtgcatttgacggaagggaagagccat

SEQ ID NO: 89 - DNA, 74 bp, Artificial Sequence
  Description of Artificial Sequence primer nifD3
gatcagctgttgaaagctattaaaccacaaaaaggattactccggcccttatcacggttacgacggatttgcta

SEQ ID NO: 90 - DNA, 74 bp, Artificial Sequence
  Description of Artificial Sequence primer nifD4
gatctagcaaatccgtcgtaaccgtgataagggccggagtaatcctttttgtggtttaatagctttcaacagct

SEQ ID NO: 91 - DNA, 41 bp, Artificial Sequence
  Description of Artificial Sequence primerSSV1-1
ataagaatgcggccgcccgatatgacgaaagataagacgcg

SEQ ID NO: 92 - DNA, 70 bp, Artificial Sequence
  Description of Artificial Sequence primer SSV1-2
ataagaatgcggccgcaccatgcccaagaagaagaggaaggtgacgaaagataagacgcgttataaatac

SEQ ID NO: 93 - DNA, 47 bp, Artificial Sequence
  Description of Artificial Sequence primer SSV2
tgtcccgggctcgaaaccggggggatccgcttgtaggggagtatccc

SEQ ID NO: 94 - DNA, 47 bp, Artificial Sequence
  Description of Artificial Sequence primer SSV3
gagcccgggacaagcggaagcggtggtggaaaagagggaactgaacg

SEQ ID NO: 95 - DNA, 33 bp, Artificial Sequence
  Description of Artificial Sequence primer SSV4
atcgctcgagtcagaccccttttagccattccg

SEQ ID NO: 96 - DNA, 40 bp, Artificial Sequence
  Description of Artificial Sequence primer SSV5
atcgttcgaaggatccgcgttaacacctaagaaggcgaag

SEQ ID NO: 97 - DNA, 38 bp, Artificial Sequence
  Description of Artificial Sequence primer SSV6
atcgttcgaaggatccgatttaatctctacatggagag

SEQ ID NO: 98 - DNA, 64 bp, Artificial Sequence
  Description of Artificial Sequence primer C31-2
ataagaatgcggccgcaccatgcccaagaagaagaggaaggtgacacaaggggttgtgaccggg

SEQ ID NO: 99 - DNA, 4831 bp, Artificial Sequence
  Description of Artificial Sequence vector pRK41
ggccgcaccatgcccaagaagaagaggaaggtgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtagcggccgctctagaactagtggatcccccgggctgcaggaattcgatatcaagcttatcgataccgtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggc

SEQ ID NO: 100 - DNA, 20 bp, Artificial Sequence
  Description of Artificial Sequence primer C31-screen 1
gcgtgagatcaagacgcaca

SEQ ID NO: 101 - DNA, 21 bp, Artificial Sequence
  Description of Artificial Sequence primer C31-screen 2
gcagcggtaagagtccttgat

SEQ ID NO: 102 - DNA, 20 bp, Artificial Sequence
  Description of Artificial Sequence primer beta-Gal 3
atcctctgcatggtcaggtc

SEQ ID NO: 103 - DNA, 18 bp, Artificial Sequence
  Description of Artificial Sequence primer beta-Gal 4
cgtggcctgattcattcc

SEQ ID NO: 104 - DNA, 5878 bp, Artificial Sequence
  Description of Artificial Sequence vector pCAGGS-Cre-pA
cgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagtaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctcgaccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgcagatccactagttctagggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggag

SEQ ID NO: 105 - DNA, 6641 bp, Artificial Sequence
  Description of Artificial Sequence vector pCAGGSC31CNLS-pA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagtaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcccgatatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgcctaagaagaagaggaaggtttagactctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttttcgac

SEQ ID NO: 106 - DNA, 11784 bp, Artificial Sequence
  Description of Artificial Sequence modified ROSA26 locus
ggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttcgcctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgacgacactgcagagacctacttcactaacaaccggtacagttcgtggaccagatgggtgaggtggagtacgcgcccggggagcccaagggcacgccctggcacccgcaccgcggcttcgagaccgtcacgaataacttcgtatagcatacattatacgaagttataagctcgatgaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttcagctgctcgactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgtgtcatgtcggcgaccctacgcccccaactgagagaactcaaaggttaccccagttggggcactactcccgaaaaccgcttctggatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccaaatcggccggcctaggcgcgccggtaaccgaagttcctatactttctagagaataggaacttcggaataggaacttcaagcttaagcgctagcctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacat

SEQ ID NO: 107 - DNA, 1458 bp, Bacteriophage TP901-1
  CDS (1)..(1455)
atgactaagaaagtagcaatctatacacgagtatccactactaaccaagcagaggaagggttctcaattgatgagcaaattgaccgtttaacaaaatatgctgaagcaatggggtggcaagtatctgatacttatactgatgctggtttttcaggggccaaacttgaacgcccagcaatgcaaagattaatcaacgatatcgagaataaagcttttgatacagttcttgtatataagctagaccgcctttcacgtagtgtaagagatactctttatcttgttaaggatgtgttcacaaaaaataaaatagactttatctcgcttaatgaaagtattgatacttcttctgctatgggtagcttgtttctcactattctttctgcaattaatgagtttgaaagagagaatataaaagaacgcatgactatgggtaaactagggcgagcgaaatctggtaagtctatgatgtggactaagacagcttttgggtattaccacaacagaaagacaggtatattagaaattgttcctttacaagctacaatagttgaacaaatattcactgattatttatcaggaatatcacttacaaaattaagagataaactcaatgaatctggacacatcggtaaagatataccgtggtcttatcgtaccctaagacaaacacttgataatccagtttactgtggttatatcaaatttaaggacagcctatttgaaggtatgcacaaaccaattatcccttatgagacttatttaaaagttcaaaaagagctagaagaaagacaacagcagacttatgaaagaaataacaaccctagacctttccaagctaaatatatgctgtcagggatggcaaggtgcggttactgtggagcacctttaaaaattgttcttggccacaaaagaaaagatggaagccgcactatgaaatatcactgtgcaaatagatttcctcgaaaaacaaaaggaattacagtatataatgacaataaaaagtgtgattcaggaacttatgatttaagtaatttagaaaatactgttattgacaacctgattggatttcaagaaaataatgactccttattgaaaattatcaatggcaacaaccaacctattcttgatacttcgtcatttaaaaagcaaatttcacagatcgataaaaaaatacaaaagaactctgatttgtacctaaatgattttatcactatggatgagttgaaagatcgtactgattcccttcaggctgagaaaaagctgcttaaagctaagattagcgaaaataaatttaatgactctactgatgtttttgagttagttaaaactcagttgggctcaattccgattaatgaactatcatatgataataaaaagaaaatcgtcaacaaccttgtatcaaaggttgatgttactgctgataatgtagatatcatatttaaattccaactcgcttaa

SEQ ID NO: 108 - protein, 485 aa, Bacteriophage TP901-1
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGITVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA